(19) United States
(12) Patent Application Publication
(10) Pub. No.: US 2006/0223729 A1
(43) Pub. Date: Oct. 5, 2006

(54) Photodynamic inactivation of bacterial spores

(76) Inventors: Michael R. Hamblin, Revere, MA (US); Tatiana N. Demidova, Cambridge, MA (US)

Correspondence Address: EDWARDS & ANGELL, LLP, P.O. BOX 55874, BOSTON, MA, 02205, US

(21) Appl. No.: 10/570,851

(22) PCT Filed: Sep. 7, 2004
(86) PCT No.: PCT/US04/28971

§ 371 Date: Apr. 5, 2006

Related U.S. Application Data
(60) Provisional application No. 60/500,431, filed on Sep. 5, 2003.

(51) Int. Cl. A61K 8/00

(52) U.S. Cl. 510/130; 510/386

(57) ABSTRACT

The present invention relates the use photosensitizers to inactivate bacterial spores of bacterial species including Bacillus anthracis. Methods of the present invention are useful in the decontamination and treatment of living animals and in the decontamination of inanimate objects and substances.

### RELATED APPLICATIONS/PATENTS & INCORPORATION BY REFERENCE

This application claims priority to U.S. Application Ser. No. 60/500,431, filed on Sep. 5, 2003 as Attorney Docket No. 910000-2053.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### STATEMENT OF RIGHTS TO INVENTION MADE UNDER FEDERALLY SPONSORED RESEARCH

This work was supported, in part, by the government by a grant from the Department of Defense as part of the Medical Free Electron Laser Program (grant DOD MFEL N 00014-94-1-0927). The government may have certain rights to this invention.

### BACKGROUND

Spore formation is a sophisticated mechanism by which some Gram positive bacteria, such as Bacillus anthracis and Bacillus cereus, survive conditions of external stress and nutrient deprivation by producing a multi-layered protective capsule enclosing their dehydrated and condensed genomic DNA (Yudkin, 1993). When such bacterial spores encounter a favorable environment, germination can take place, enabling the bacteria to reproduce and, in the case of pathogenic species, cause disease. Bacterial spores possess a coat and membrane structure that is highly impermeable to most molecules that could be toxic to the dormant bacteria (Driks, 2002). Therefore, spores are highly resistant to damage by heat, radiation, and many of the commonly employed anti-bacterial agents, and can only be destroyed by some severe chemical procedures including oxidizing vapors such as peracetic acid, chlorine dioxide and ozone, and DNA cross-linking vapors such as ethylene oxide and glutaraldehyde (Russell, 1990; Whitney et al., 2003). Multiple bacterial species employ this spore forming mechanism, including several medically important pathogens of the Bacillus and Clostridium genera.

Bacillus anthracis (“B. anthracis”) is the pathogenic organism that causes anthrax—a disease which is frequently fatal due to the ability of this bacterium to produce deadly toxins (Chaudry et al., 2001). Using experimental anthrax in the 1870s, Robert Koch demonstrated for the first time the bacterial origin of a specific disease, and also discovered the spore stage that allows persistence of the organism in the environment. Shortly afterward, B. anthracis was recognized as the cause of inhalational anthrax. One route of anthrax infection is through entry of B. anthracis spores into cuts and abrasions in the skin. Infection by this route causes the serious, but usually not fatal disease, cutaneous anthrax (Tutrone et al., 2002). On the other hand, infection through inhalation of B. anthracis spores (“inhalational anthrax”) is frequently fatal. In addition, B. anthracis infection can also be caused by the ingestion of contaminated material (“gastrointestinal anthrax”).

In nature, infection of humans with anthrax is usually caused by exposure to spores from infected livestock or contaminated animal products. However, in recent years concerns have grown about non-natural exposure routes, for example exposure as the result of deliberate release of B. anthracis spores in biological warfare and bio-terrorism (Spencer & Lightfoot, 2001).

In the second half of this century, anthrax was developed as part of a larger biological weapons program by several countries. B. anthracis spores can be “weaponized” in a laboratory by milling spores into a dry powder of a sufficiently small particle size that enables aerosol dispersal of the spores (Wiener, 1996). The World Health Organization estimated that 50 kg of B. anthracis spores released upwind of a population center of 500,000 would result in up to 95,000 fatalities, with an additional 125,000 persons incapacitated (Huxsoll, D. L. et al., JAMA 262:677-679 (1989)).

A later analysis, by the Office of Technology Assessment of the U.S. Congress, estimated that 130,000 to 3 million deaths could occur following the release of 100 kilograms of aerosolized anthrax over Washington D.C. Dispersal experiments with the simulant Bacillus globigii in the New York subway system in the 1960s suggested that release of a similar amount of B. anthracis during rush hour would result in 10,000 deaths.

The largest experience with inhalation anthrax occurred after the accidental release of aerosolized anthrax spores in 1979 at a military biology facility in Sverdlovsk, Russia when 79 cases of inhalation anthrax were reported, 68 of those being fatal. More recently, instances of anthrax contaminated mail in the U.S. highlighted the danger of exposure to even a small number of spores (Dull et al., 2002).

The deliberate release of B. anthracis spores has the ability to cause major devastation. Thus, effective methods of diagnosis and treatment are of vital importance. One of the characteristics of anthrax infection that causes particular problems for disease management is its variable and sometimes long incubation period. Exposure to an aerosol of anthrax spores could cause symptoms as soon as 2 days after exposure or as late as 6-8 weeks after exposure (in Sverdlovsk one case developed 46 days after exposure). Furthermore, the early symptoms of anthrax infection are rather non-specific (typically consisting of fever and/or a cough) and in most cases death occurs within 1-3 days of the onset of these symptoms. Because most antibiotics are only effective if treatment is started before the development of symptoms, early detection and diagnosis are vital.

Following the deliberate dissemination of B. anthracis spores through the U.S. mail in 2002, public health officials were faced with two major problems: detecting spores in buildings and on exposed individuals, and treating those people thought to be exposed and the few who actually became infected. Thousands of people who were thought to have been exposed were treated with antibiotics, usually ciprofloxacin. Fortunately, those undergoing preventative treatment did not become infected; the intervention was effective because the particular strain used in the attack was wholly susceptible to the usual antibiotics.

However, the situation could have been much worse if the strain had been resistant to antibiotics. Experts agree that such multi-antibiotic resistant B. anthracis spores could be readily created by competent microbiologists using transfection with plasmids carrying multiple resistance genes (Gilligan, 2002). Were such spores to be released on the battlefield or in a terrorist attack, the only defense would be vaccination of personnel or protection against contact with the spores. Although protective suits and respirators would undoubtedly be used by military personnel when a likelihood of spore release was considered, during warfare the additional use of conventional weapons such as firearms and explosives could still create wounds that would be readily contaminated with spores. In the case of the release of anthrax spores during a terrorist attack, it is likely that many people would not have access to such protective suits.

The U.S. has a sterile protein-based human anthrax vaccine that was licensed in 1970 and has been mandated for use by all U.S. military personnel. However, the present anthrax vaccine is less than 100% effective (Chaudry et al., 2001; Kimmel et al., 2003; Lutwick & Nierengarten, 2002). Furthermore, because vaccine supplies are limited and production capacity is modest, there is currently no vaccine available for civilian use.

Concerns about antibiotic resistance and the lack of a widely available vaccine have spurred intense research into alternative forms of preventing and treating B. anthracis infection. Effective and more acceptable vaccines are being developed. However, these, like many other vaccines, will require multiple immunizations and time for protection to build up. To be effective, a vaccine would need to be administered well in advance of an attack.

Another attractive possibility is the use of sporicidal agents. However currently available sporicidal agents are too toxic to be introduced into wounds or applied to mucous membranes. Thus, there is a pressing need for the development of alternative non-vaccine, non-antibiotic methods to control infections caused by spore forming organisms, such as anthrax infection.

Photodynamic therapy, or PDT, has received regulatory approval for several indications/diseases including cancer (Dougherty et al., 1998). Its use as a cancer treatment is based on the observation that certain non-toxic dyes known as photosensitizers, (“PS”) of which hematoporphyrin derivative (“HPD”, also known as Photofrin) is the best known example, accumulate preferentially in malignant tissues (Hamblin & Newman, 1994). Therapy involves delivering visible light of the appropriate wavelength to excite the PS molecule to the excited singlet state. This excited state may then undergo intersystem crossing to the slightly lower energy triplet state, which can then react further by one or both of two pathways known as Type I and Type II photo-processes, both of which require oxygen (Ochsner, 1997).

The Type I pathway involves electron transfer reactions from the PS triplet state with the participation of a substrate to produce radical ions which can then react with oxygen to produce cytotoxic species such as superoxide, hydroxyl and lipid derived radicals (Athar et al., 1988). The Type II pathway involves energy transfer from the PS triplet state to ground state molecular oxygen (triplet) to produce the excited state singlet oxygen, which can then oxidize many biological molecules such as proteins, nucleic acids and lipids, and lead to cytotoxicity (Redmond & Gamlin, 1999).

Although originally developed as a cancer treatment, the most successful PDT application to date, which is now FDA approved, is an ophthalmological treatment for age-related macular degeneration (Bressler & Bressler, 2000; Henney, 2000). Other non-oncological applications of PDT at a less developed stage include treatments for psroriasis (Boehncke et al., 2000), arthritis (Trauner & Hasan, 1996), Barretts's esophagus (Barr, 2000), acne (Hongcharu et al., 2000), atherosclerosis (Rockson et al., 2000) and restenosis (Jenkins et al., 1999) in both veins and arteries.

Most of the PS that are under investigation for the treatment of cancer and other tissue diseases are based on the tetrapyrrole nucleus. Examples are porphyrins (“HPD”), chlorins (“BPD”), bacteriochlorins, phthalocyanines, and naphthalocyanines (Boyle & Dolphin, 1996). These molecules have been chosen for their low dark toxicity to mammalian cells and to animals, and for their tumor-localizing properties. However many other PS have different molecular frameworks. These include halogenated xanthenes such as Rose Bengal (Schafer et al., 2000), phenothiaziniums such as toluidine blue (Bhatti et al., 1998), acridines (Hass & Webb, 1981) psoralens (de Mol et al., 1981) and perylenequinones such as hypericin (Kubin et al., 1999). Martin et al (Martin & Logsdon, 1987) investigated a set of thiazine, xanthene, acridine, and phenazine dyes and their phototoxicity towards E coli and concluded that oxygen radicals were primarily responsible for the toxicity of the dyes examined.

It has long been known that certain microorganisms can be killed by the combination of dyes and light in vitro (Hausmann, 1908; Jesionek & von Tappenier, 1903; Raab, 1900; Von Tappeiner & Jodlbauer, 1904), The use of photosensitizers and light to kill or inactivate microorganisms is known as “photodynamic inactivation” or “PDI.” In the 1990s, it was observed that there was a fundamental difference in susceptibility to PDI between Gram (+) and Gram (−) bacteria. It was found that in general, neutral or anionic PS molecules are efficiently bound to, and photodynamically inactivate, Gram (+) bacteria, whereas they are bound only to the outer membrane of Gram (−) bacterial cells and do not necessarily inactivate such cells after irradiation (Malik et al., 1992).

The high susceptibility of Gram (+) species is explained by their physiology, as their cytoplasmic membrane is surrounded by a relatively porous layer of peptidoglycan and lipoteichoic acid that allows photosensitizers, such as deuteroporphyrin (“DP”), to cross the membrane (Malik et al., 1992). Several groups later devised approaches that would allow PDI of Gram (−) species. Nitzan et al. (1992) used the polycationic peptide polymyxin B nonapeptide (“PMBN”), which increases the permeability of the Gram (−) outer membrane and allows PS that are normally excluded from the cell to penetrate to a location where the reactive oxygen species generated upon irradiation executes fatal damage. Malik et al. used a mixture of hemin and DP as a PDI agent against Staphylococcus aureus (“S. aureus”) and other Gram (+) bacteria (Malik et al., 1990). A similar approach was taken by Bertoloni et al (Bertoloni et al., 1990), who found that the use of Tris-ethylenediamine tetra-acetic acid (EDTA) to release lipopolysaccharide (“LPS”) or the induction of competence with calcium chloride sensitized Eschericia coli and Klebsiella pneumoniae to PDI by hematoporphyrin or zinc phthalocyanine.

A second approach adopted by several groups was to use a PS molecule with an intrinsic positive charge. Wilson and co-workers used the phenothiazinium toluidine blue O to carry out PDI of a large range of Gram (+) and Gram (−) bacteria (Bhatti et al., 1998) including S. aureus (Wilson & Yianni, 1995) and the Gram (−) bacterium Helicobacter pylori (Millson et al., 1996). Jori et al. used cationic porphyrins (meso-tetra (N-methyl)-4-pyridyl)-porphine tetraiodide and tetra-(4N,N,N-trimethyl-anilinium)-porphine to photoinactivate Gram (−) species such as Vibrio anguillarum and E. coli (Merchat et al., 1996a; Merchat et al., 1996b). Intrestingly, they also found that incubation with cationic phthalocyanines in the dark led to increased sensitivity of the bacteria to hydrophobic but not hydrophilic antibiotics.

There are some reports of PDI of Gram (−) bacteria in which it is clear that the PS does not have to penetrate the bacterium to be effective, or indeed even come into contact with the cells. According to these reports, if singlet oxygen can be generated in sufficient quantities near to the bacterial outer membrane it will be able to diffuse into the cell to inflict damage on vital structures (Dahl et al., 1987). In one set of studies, the bacteria were separated from the PS by a layer of moist air, and singlet oxygen in the gas phase diffused across the gap before contacting the bacteria (Dahl et al., 1989). In another study, the PS Rose Bengal was covalently bound to small polystyrene beads that were allowed to mix with the bacteria in suspension (Bezman et al., 1978).

Some targeting systems for PDI of bacteria presumably also rely on the ability of PS bound at the outer membrane to generate reactive oxygen species that then diffuse into the cells. For example, Yarmush et al. (Friedberg et al., 1991; Lu et al., 1992) used a PS covalently bound to a monoclonal antibody (“Mab”) that recognizes cell surface antigens expressed on Pseudomonas aeruginosa, and demonstrated specific killing of target bacteria after irradiation that was not shown by non-specific Mab conjugates. Other studies used a non-specific IgG recognized by protein A expressed on S. aureus (Gross et al., 1997). Because it is very unlikely that covalent antibody bound PS could penetrate the outer membrane, diffusion of reactive oxygen species inwards to the interior of the cell was presumably occurring in these studies (the diffusion distance of singlet oxygen in solution has been estimated to be approximately 50 nm (Ochsner, 1997)).

The failure of some PS that bind to Gram (−) species to produce any killing, indicates that reactive species produced on irradiation are not always able to diffuse inward to sensitive sites. It is now hypotheised that photosensitizers that operate chiefly via Type I mechanisms need to penetrate the outer membrane of Gram (−) bacteria in order to work, while those that act mainly by Type II mechanisms can be effective in PDI without penetrating the outer membrane.

Two basic mechanisms have been proposed to account for the lethal damage caused to bacteria by PDI: (a) DNA damage, and (b) damage to the cytoplasmic membrane. There is much evidence that treatment of bacteria with various photosensitizers and light leads to DNA damage. Both single and double DNA-strand break and the disappearance of the plasmid supercoiled fraction have been detected in Gram (+) and Gram (−) species after PDI with a wide range of PS structural types (Brendel, 1973; Harrison et al., 1972; Jacob, 1971; Jacob et al., 1977; Ziebell et al., 1977).

However, various authors have concluded that, although DNA damage occurs, it may not be the prime cause of bacterial cell death. Thus, Deinococcus radiodurans, which is known to have a very efficient DNA repair mechanism, is easily killed by PDI (Schafer et al., 2000). The alteration of cytoplasmic membrane proteins by PDI has been shown by Valduga et al (Valduga et al., 1999) and Bertoloni et al (Bertoloni et al., 1990). The disturbance of cell-wall synthesis and the appearance of multilamellar structures near the septum of dividing bacterial cells, along with loss of potassium ions from the cells, has also been reported (Nitzan et al., 1992).

Thus, there are many studies showing that photosensitizers can be effectively used in photodynamic inactivation of vegetative bacterial cells. However, to date there have been no reports of the successful use of PDI to inactivate or destroy bacterial spores. Rather, it has been shown that spores are resistant to photodynamic inactivation using dyes that easily destroy the vegetative stages of the bacteria from which the spores are generated. For example, it has been shown that Bacillus spores are resistant to photoinactivation (Schafer et al., 2000). This is not surprising given the fact that an identifying characteristic of bacterial spores is that they are extremely resistant to destruction by heat, radiation, pressure, and chemicals.

### OBJECT AND SUMMARY OF THE INVENTION

The present invention provides methods for the use of photosensitizer compositions to destroy bacterial spores, including those of Bacillus anthracis. Methods of the present invention are useful in the de-contamination and treatment of living animals, inanimate objects or substances containing unwanted spores.

It has now been shown that spores of several bacterial species including but not limited to those of B. anthracis, Bacillus cereus (“B. cereus”), Bacillus thuringiensis (“B. thuringiensis”), Bacillus subtilis (“B. subtilis”), and Bacillus atrophaeus (“B. atrophaeus”) can be destroyed using photosensitizer compositions.

Accordingly, in one aspect, the present invention provides a method of inactivating bacterial spores comprising contacting the bacterial spores with a photosensitizer composition and irradiating the bacterial spores such that a phototoxic species is produced that inactivates the bacterial spores. The bacterial spores to be inactivated include those produced by bacteria of the genus Bacillus, Clostridium, Methylosinus, Azotobacter, Bdellovibrio, Myxococcus, Cyanobacteria, Thermoactinomyces, Myxococcus, Desulfotomaculum, Marinococcus, Sporosarcina, Sporolactobacillus and Oscillospira.

In one aspect, the present invention provides methods for the inactivation of bacterial spores in or on a living animal, such as a human. The bacterial spores can be located, for example, on the skin, hair or mucous membranes of the animal. In a specific embodiment, the bacterial spores may penetrate the outermost protective epithelia of the animal, for example through wounds, cuts or abrasions in the skin or mucous membranes of the animal.

In one embodiment, the present invention provides a method of treating a subject contaminated with bacterial spores, said method comprising the steps of administering a photosensitizer to the subject, irradiating the subject such that a phototoxic species is produced that inactivates the bacterial spores, thereby treating the subject.

In another aspect, the present invention provides methods for the inactivation of bacterial spores found in inanimate substances and objects, such as animal-derived products, biological fluids, food, water, air, hard-surfaces, equipment, and machinery and clothing.

Various photosensitizers can be used in conjunction with methods of the present invention. In one embodiment, the photosensitizers include but are not limited to Phenothiazinium dyes, phenodiazinium dyes, or phenooxazinium dyes. In specific embodiments the photosensitizers include but are not limited to toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine 0, neutral red, 5-ethylamino-9-diethylaminobenzo[a]phenothiazinium chloride, 5-ethylamino-9-diethylaminobenzo[a]phenoselenazinium chloride, thiopyronine, and thionine.

In certain embodiments the photosensitizers of the present invention are formulated in compositions that also contain one or more additional agents such as pharmaceutically acceptable carriers, excipients, antibiotics, sporicidal agents, disinfectants, or detergents. In other embodiments, photosensitizers of the present invention are co-administered with pharmaceutically acceptable carriers, excipients, antibiotics, sporicidal agents, disinfectants, or detergents, optionally present within the same composition as the photosensitizer.

In specific embodiments, irradiation is provided by a light source that emits light having a wavelength in the range of about 450 to about 750 nm and/or with a fluence in the range of about 10 to about 1000 J/cm². Such a light source can be, for example, natural sunlight, a lamp, a laser or a fiber optic device.

Other objects and advantages of the present invention will be apparent from the detailed description that follows.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings, in which:

FIG. 1 depicts a graph showing the effects of treatment of B. cereus spores with 100 μM of toluidine blue O. As described in Example 1, the duration of toluidine blue O treatment was 10 minutes, following which B. cereus spores were irradiated with a fluence rate of 100 mW/cm² 635-nm light at various fluences ranging from 0 to 500 J/cm².

FIG. 2 depicts a graph illustrating the effects of 10 μM, 100 μM and 1 mM toluidine blue O on the survival of B. cereus spores. Spores were incubated with toluidine blue O for 10 minutes and irradiated with a fluence rate of 100 mW/cm² 635-nm light at various fluences ranging from 0 to 300 J/cm².

FIG. 3 depicts a graph showing the effect of toluidine blue O at stated concentrations on the survival of spores of B. cereus, B. thuringiensis, B. subtilis and B. atrophaeus irradiated at a fluence rate of 100 mW/cm² with 635-nm light at various fluences ranging from 0 to 300 J/cm².

FIG. 4 depicts a graph showing the effect of spore concentration on the efficiency of toluidine blue O-mediated spore inactivation/killing. Samples of B. cereus spores at concentrations of 10⁷ spores/mL and 10⁶ spores/mL were exposed to 100 μM toluidine blue O for 30 minutes followed by irradiation with a fluence rate of 100 mW/cm² 635-nm light at various fluences ranging from 0 to 15 j/cm².

FIG. 5 depicts a graph showing survival of B. cereus spores following treatment with 100 μM toluidine blue O, 100 μM AzureA, 100 μM AzureB, and 100 μM Azure C for 1 hour followed by irradiation with a fluence rate of 100 mW/cm² appropriate wavelength light at various fluences ranging from 0 to 32 J/cm².

FIG. 6 depicts a graph showing survival of B. cereus spores following treatment with 50 μM toluidine blue O for various times followed by irradiation with a fluence rate of 100 mW/cm² appropriate wavelength light at various fluences ranging from 0 to 32 J/cm².

FIG. 7 depicts a graph showing survival of B. cereus spores following treatment with 50 μM dimethylmethylene blue for various times followed by irradiation with a fluence rate of 100 mW/cm² 670 nm light at various fluences ranging from 0 to 32 J/cm².

FIG. 8 depicts a graph showing survival of B. cereus spores following treatment with 100 μM of each of dimethylmethylene blue, new methylene blue, safranin O, methylene blue violte 3RAX, toluidine blue O, and malachite green for 1 hour followed by irradiation with a fluence rate of 100 mW/cm² appropriate wavelength light at various fluences ranging from 0 to 32 J/cm².

FIG. 9 depicts a graph showing survival of B. thuringiensis spores following treatment with 100 μM of each of dimethylmethylene blue, new methylene blue, and toluidine blue O for 1 hour followed by irradiation with a fluence rate of 100 mW/cm² appropriate wavelength light at various fluences ranging from 0 to 32 J/cm².

FIG. 10 depicts a graph showing survival of B. thuringiensis spores following treatment with 100 μM of each of Azure A, Azure B, and Azure C, for 1 hour followed by irradiation with a fluence rate of 100 mW/cm² appropriate wavelength light at various fluences ranging from 0 to 32 J/cm².

FIG. 11 depicts a graph showing survival of B. subtilis (labeled Bs) and B. atrophaeus (labeled Ba) spores following treatment with 100 μM toluidine blue O for 24 hours, or 1 mM toluidine blue O for 1 hour or 10 minutes followed by irradiation with a fluence rate of 100 mW/cm² 635-nm light at various fluences ranging from 0 to 300 J/cm².

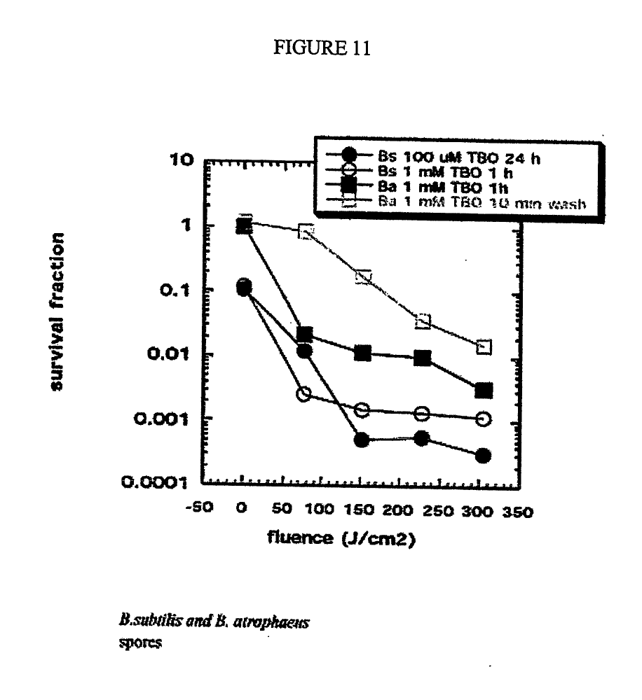

FIG. 12 depicts a graph showing the survival fraction of B. cereus spores following photodynamic treatment with two isosteric dyes.

FIG. 13 depicts a graph showing the survival fraction of B. cereus and B. subtilis spores and vegetative cells following photodynamic treatment with toludine blue.

Other aspects of the invention are described in or are obvious from the following disclosure, and are within the ambit of the invention.

### DETAILED DESCRIPTION

I. Definitions

As used herein, the following terms have the meanings ascribed to them unless specified otherwise. As used herein, the terms “comprises”, “comprising”, and the like can have the meaning ascribed to them in U.S. Patent Law and can mean “includes”, “including” and the like.

The term “bacterial spore” as used herein has its normal meaning which is well known and understood by those of skill in the art. A “bacterial spore” is a form of a bacterial cell which has protective structural features and reduced metabolic activity such that it can survive adverse growth conditions for extended periods of time. The term “spore” includes endospores, exospores and cysts.

“Inactivation” as used herein refers to any method of killing, destroying, or otherwise functionally incapacitating a bacteria contained in a spore. Thus, a bacterial spore that is “inactivated” is one in which the bacteria within has been killed, destroyed, or otherwise functionally incapacitated.

The term “sporicidal agent”, as used herein refers to any agent capable of inactivating a bacterial spore.

The terms “photosensitizer “P S” and “photosensitive dye” are used herein refer to chemical compounds, or biological precursors thereof, that are “activated” (or “photoactivated”) by irradiation with light of a particular wavelength or range of wavelengths to produce “reactive species” or “phototoxic species.” Such reactive species are chemical species (e.g., free radicals) that are toxic to cells, such as bacterial cells and bacterial cells within spores. Photosensitizer compositions that are capable of inactivating bacterial spores can also be referred to as “photosensitive sporicidal agents” or “photodynamic sporicidal agents.”

As used herein, a “photosensitizer composition” or “photosensitive dye composition” is any composition that comprises a photosensitizer.

The term “irradiate” can be used interchangeably with the term “illuminate” to mean providing light at a desired wavelength and fluence rate.

A “subject” is a vertebrate, preferably a mammal, more preferably a human. Mammals include, but are not limited to, humans, animals (farm animals, sport animals, and pets).

The term “photodynamic therapy” (or “PDT”) as used herein refers to processes and methods by which photosensitizers can be used to bring about some therapeutically beneficial effect. The term “photodynamic inactivation” (“PDI”) as used herein refers to processes and methods by which photosensitizers can be used to inactivate cells, including bacterial cells and bacterial spores, to either a) bring about some therapeutically beneficial effect in a living animal or b) decontaminate a living animal, a substance or an inanimate object.

The term “decontaminate” as used herein refers to the process of inactivating bacterial cells or spores, and can be used interchangeably with the terms “disinfect” and “sterilize.” The terms “inanimate substance” and “inanimate object,” as used herein mean any material thing that is not a whole living animal, and includes materials comprising or consisting of solids, liquids and gases. “Substances” and “objects” can consist of or comprise living material such as plants and parts of animals such as isolated animal tissues or cells.

As used herein the term “administer” means to contact with, apply, give, deliver, or treat a living animal or an object or substance with a photosensitizer composition.

Further definitions may appear in context throughout the disclosure provided herein.

II. Methods of the Invention

In one embodiment, methods of the present invention are directed to the decontamination and/or treatment of living animals, such as humans, that have come into contact with bacterial spores. In another embodiment, methods of the present invention are directed to the disinfection of substances and objects that have come into contact with bacterial spores.

A. Decontamination and/or Treatment of Living Animals

Methods of the present invention provide a means for treating or decontaminating living animals that have, or may have, come into contact with bacterial spores. Methods of the invention can be performed by contacting the living animal that has been contaminated (or is suspected of being contaminated) with a photosensitizer composition and irradiating the photosensitizer composition with a light source that emits light at an effective wavelength and fluence rate (i.e., an “effective light source”). In so doing, bacterial spores in or on the living animal will be inactivated.

If the bacterial spores are suspected of being located at a particular location in or on a living animal, the application of the photosensitizer and the irradiation with an effective light source can be targeted to that area For example, wounds, cuts and abrasions in the skin may be targeted by direct application of the photosensitizer composition to that area. In addition, mucous membranes such as those in the respiratory tract may be targeted for decontamination. Alternatively, the whole living animal can be treated with the photosensitizer composition, through, for example, oral or topical administration, followed by irradiation with an effective light source throughout the body.

In a specific embodiment, the living animals that are decontaminated using methods of the present invention are humans. A particular advantage of the present invention is that the photosensitizers are non-toxic when the irradiation and/or amount of photosensitizer is provided in controlled doses and therefore safe for human use.

Bacterial spores to be inactivated can be those of any bacterial species known in the art that produces spores. In one embodiment, the contaminating bacterial spores to be inactivated are those produced by bacteria of the genus Bacillus. In specific embodiments, the bacterial spores to be inactivated include Bacillus acidocaldarius, Bacillus acidoterrestris, Bacillus aeolius, Bacillus agaradhaerens, Bacillus agri, Bacillus alcalophilus, Bacillus alginolyticus, Bacillus alvei, Bacillus amyloliquefaciens, Bacillus amylolyticus, Bacillus aneurinilyticus, Bacillus anthracis, Bacillus aquimaris, Bacillus arseniciselenatis, Bacillus atrophaeus, Bacillus azotofixans, Bacillus azotoformans, Bacillus badius, Bacillus barbaricus, Bacillus bataviensis, Bacillus benzoevorans, Bacillus borstelensis, Bacillus brevis, Bacillus carboniphilus, Bacillus centrosporus, Bacillus cereus, Bacillus chitinolyticus, Bacillus chondroitinus, Bacillus choshinensis, Bacillus circulans, Bacillus clarkii, Bacillus clausii, Bacillus coagulans, Bacillus cohnii, Bacillus curdlanolyticus, Bacillus cycloheptanicus, Bacillus decolorationis, Bacillus dipsosauri Bacillus drentensis, Bacillus edaphicus, Bacillus ehimensis, Bacillus endophyticus, Bacillus farraginis, Bacillus fastidiosus, Bacillus firmus, Bacillus flexus, Bacillus fordii Bacillus formosus, Bacillus fortis, Bacillus fumarioli Bacillus funiculus, Bacillus fusiformis, Bacillus galactophilus, Bacillus galactosidilyticus, Bacillus gelatini, Bacillus gibsonii, Bacillus globisporus, Bacillus globisporus, Bacillus globisporus subspecies marinus, Bacillus glucanolyticus, Bacillus gordonae, Bacillus halmapalus, Bacillus haloalkaliphilus, Bacillus halodenitrificans, Bacillus halodurans, Bacillus halophilus, Bacillus horikoshii, Bacillus horti, Bacillus hwajinpoensis, Bacillus indicus, Bacillus infernos, Bacillus insolitus, Bacillus jeotgali, Bacillus kaustophilus, Bacillus kobensis, Bacillus krulwichiae, Bacillus larvae, Bacillus laterosporus, Bacillus lautus, Bacillus lentimorbus, Bacillus lentus, Bacillus licheniformis, Bacillus luciferensis, Bacillus macerans, Bacillus macquariensis, Bacillus marinus, Bacillus marisflavi, Bacillus marismortui, Bacillus megaterium, Bacillus methanolicus, Bacillus migulanus, Bacillus mojavensis, Bacillus mucilaginosus, Bacillus mycoides, Bacillus naganoensis, Bacillus nealsonii, Bacillus neidei, Bacillus niacini, Bacillus novalis, Bacillus odysseyi, Bacillus okuhidensis, Bacillus oleronius, Bacillus pabuli, Bacillus pallidus, Bacillus pantothenticus, Bacillus parabrevis, Bacillus pasteurii, Bacillus peoriae, Bacillus polymyxa, Bacillus popilliae, Bacillus pseudalcaliphilus, Bacillus pseudofirmus, Bacillus pseudomycoides, Bacillus psychrodurans, Bacillus psychrophilus, Bacillus psychrosaccharolyticus, Bacillus psychrotolerans, Bacillus pulvifaciens, Bacillus pumilus, Bacillus pycnus, Bacillus reuszeri, Bacillus salexigens, Bacillus schlegelii, Bacillus selenitireducens, Bacillus shackletonii, Bacillus silvestris, Bacillus simplex, Bacillus siralis, Bacillus smithii, Bacillus soli, Bacillus sonorensis, Bacillus sphaericus, Bacillus sporothermodurans, Bacillus stearothermophilus, Bacillus subterraneus, Bacillus subtilis, Bacillus subtilis subspecies spizizenii, Bacillus subtilis, Bacillus thermantarcticus, Bacillus thermoaerophilus, Bacillus thermoamylovorans, Bacillus thermocatenulatus, Bacillus thermocloacae, Bacillus thermodenitrificans, Bacillus thermoglucosidasius, Bacillus thermoleovorans, Bacillus thermoruber, Bacillus thermosphaericus, Bacillus thiaminolyticus, Bacillus thuringiensis, Bacillus tusciae, Bacillus validus, Bacillus vallismortis, Bacillus vedderi, Bacillus vireti, Bacillus vulcani and Bacillus weihenstephanensis.

In another embodiment, the bacterial spores to be inactivated are those produced by bacteria of the genera Clostridium. In specific embodiments, the bacterial spores to be inactivated include Clostridium absonum, Clostridium aceticum, Clostridium acetireducens, Clostridium acetobutylicum, Clostridium acidisoli, Clostridium acidurici, Clostridium aerotolerans, Clostridium akagii, Clostridium aldrichii, Clostridium algidicarnis, Clostridium algidixylanolyticum, Clostridium aminophilum, Clostridium aminovalericum, Clostridium amygdalinum, Clostridium arcticum, Clostridium argentinense, Clostridium aurantibutyricum, Clostridium baratii Clostridium barkeri, Clostridium beijerinckii, Clostridium bifermentans, Clostridium bolteae, Clostridium botulinum, Clostridium bowmanii, Clostridium bryantii, Clostridium butyricum, Clostridium cadaveris, Clostridium caminithermale, Clostridium carnis, Clostridium, celatum, Clostridium celerecrescens, Clostridium cellobioparum, Clostridium cellulofermentans, Clostridium cellulolyticum, Clostridium cellulose, Clostridium cellulovorans, Clostridium chartatabidum, Clostridium chauvoei, Clostridium clostridioforme, Clostridium coccoides, Clostridium cochlearium, Clostridium cocleatum, Clostridium colicanis, Clostridium colinum, Clostridium collagenovorans, Clostridium cylindrosporum, Clostridium difficile, Clostridium diolis, Clostridium disporicum, Clostridium durum, Clostridium estertheticum, Clostridium estertheticum, subspcies Estertheticum, Clostridium estertheticum subspecies laramiense, Clostridiumfallax, Clostridiumfelsineum, Clostridiumfervidum, Clostridiumfimetarium, Clostridiumformicaceticum, Clostridiumfrigidicarnis, Clostndiumfrigoris, Clostridiumgasigenes, Clostridiumghonii, Clostridium glycolicum, Clostridium grantii, Clostridium haemolyticum, Clostridium halophilum, Clostridium hastiforme, Clostridium hathewayi, Clostridium herbivorans, Clostridium hiranonis, Clostridium histolyticum, Clostridium homopropionicum, Clostridium hungatei, Clostridium hydroxybenzoicum, Clostridium hylemonae, Clostridium indolis, Clostridium innocuum, Clostridium intestinale, Clostridium irregulare, Clostridium isatidis, Clostridiumjosui, Clostridium kluyveri, Clostridium lactatifermentans, Clostridium lacusfryxellense, Clostridium laramiense, Clostridium lentocellum, Clostridium lentoputrescens, Clostridium leptum, Clostridium limosum, Clostridium litorale, Clostridium lituseburense, Clostridium ljungdahlii, Clostridium lortetii, Clostridium magnum, Clostridium malenominatum, Clostridium mangenotii, Clostridium mayombei, Clostridium methoxybenzovorans, Clostridium methylpentosum, Clostridium neopropionicum, Clostridium nexile, Clostridium novyi, Clostridium oceanicum, Clostridium orbiscindens, Clostridium oroticum, Clostridium oxalicum, Clostridium papyrosolvens, Clostridium paradoxum, Clostridium paraperfringens, Clostridium paraputrificum, Clostridium pascui, Clostridium pasteurianum, Clostridium peptidivorans, Clostridium perenne, Clostridium perfringens, Clostridium pfennigii, Clostridium phytofermentans, Clostridium piliforme, Clostridium polysaccharolyticum, Clostridium populeti, Clostridium propionicum, Clostridium proteoclasticum, Clostridium proteolyticum, Clostridium psychrophilum, Clostridium puniceum, Clostridium purinilyticum, Clostridium putrefaciens, Clostridium putrificum, Clostridium quercicolum, Clostridium quinii, Clostridium ramosum, Clostridium rectum, Clostridium roseum, Clostridium saccharobutylicum, Clostridium saccharolyticum, Clostridium saccharoperbutylacetonicum, Clostridium sardiniense, Clostridium sartagoforme, Clostridium scatologenes, Clostridium scindens, Clostridium septicum, Clostridium sordellii, Clostridium sphenoides, Clostridium spiroforme, Clostridium sporogenes, Clostridium sporosphaeroides, Clostridium stercorarium, Clostridium stercorarium subspecies leptospartum, Clostridium stercorarium subspecies stercorarium, Clostridium stercorarium subspecies thermolacticum, Clostridium sticklandii, Clostridium subterminale, Clostridium symbiosum, Clostridium termitidis, Clostridium tertium, Clostridium tetani, Clostridium tetanomorphum, Clostridium thermaceticum, Clostridium thermautotrophicum, Clostridium thermoalcaliphilum, Clostridium thermobutyricum, Clostridium thermocellum, Clostridium thermocopriae, Clostridium thermohydrosulfuricum, Clostridium thermolacticum, Clostridium thermopalmarium, Clostridium thermopapyrolyticum, Clostridium thermosaccharolyticum, Clostridium thermosuccinogenes, Clostridium thermosulfurigenes, Clostridium thiosulfatireducens, Clostridium tyrobutyricum, Clostridium uliginosum, Clostridium ultunense, Clostridium, villosum, Clostridium vincentii, Clostridium viride, Clostridium xylanolyticum, and Clostridium xylanovorans.

In another embodiment, the bacterial spores to be inactivated are those produced by bacteria of the genera Myxococcus. In specific embodiments, the bacterial spores to be inactivated include Myxococcus coralloides, Myxococcus disciformis, Myxococcus flavescens, Myxococcus fulvus, Myxococcus macrosporus, Myxococcus stipitatus Myxococcus virescens, and Myxococcus xanthus.

In another embodiment, the bacterial spores to be inactivated are those produced by bacteria of the genera Desulfomaculum. In specific embodiments, the bacterial spores to be inactivated are Desulfotomaculum acetoxidans, Desulfotomaculum aeronauticum, Desulfotomaculum alkaliphilum, Desulfotomaculum auripigmentum, Desulfotomaculum australicum, Desulfotomaculum geothermicum, Desulfotomaculum gibsoniae, Desulfotomaculum guttoideum, Desulfotomaculum halophilum Desulfotomaculum kuznetsovii, Desulfotomaculum luciae, Desulfotomaculum nigriflcans, Desulfotomaculum orientis, Desulfotomaculum putei, Desulfotomaculum ruminis, Desulfotomaculum sapomandens, Desulfotomaculum solfataricum, Desulfotomaculum thermoacetoxidans, Desulfotomaculum thermobenzoicum subspecies thermobenzoicum, Desulfotomaculum thermobenzoicum subspecies thermosyntrophicum, Desulfotomaculum thermocisternum and Desulfotomaculum thermosapovorans.

In another embodiment, the bacterial spores to be inactivated are those produced by bacteria of the genera Thermoactinomyces. In specific embodiments, the bacterial spores to be inactivated are Thermoactinomyces candidus, Thermoactinomyces dichotomicus, Thermoactinomyces intermedius, Thermoactinomyces peptonophilus, Thermoactinomyces putidus, Thermoactinomyces sacchari, Thermoactinomyces thalpophilus and Thermoactinomyces vulgaris.

In another embodiment, the bacterial spores to be inactivated are those produced by bacteria of the genera Methylosinus, Azotobacter, Bdellovibrio, Cyanobacteria, Marinococcus, Sporosarcina, Sporolactobacillus, and Oscillospira.

Bacterial spores to be inactivated by methods of the invention are generally resistant to the lethal effects of heat, drying, freezing, chemicals and radiation. Types of bacterial spores can have various sub-classifications based on their physiological properties. Endospores are produced by bacteria of the genera Bacillus, Clostridium, Thermoactinomyces, Myxococcus, Marinococcus, Sporosarcina, and Oscillospira, exospores are produced by bacteria of the genera Methylosinus and cysts are produced by bacteria of the genera Azotobacter, Bdellovibrio, Myxococcus, and Cyanobacteria.

B. Decontamination of Substances and Objects

Methods of the present invention provide a means for sterilizing or decontaminating inanimate objects and substances that have, or may have, come into contact with bacterial spores. This is performed by contacting the objects that are contaminated (or are suspected of being contaminated) with a photosensitizer composition and irradiating the photosensitizer composition with a light source that emits light at an effective wavelength and fluence rate (i.e., an “effective light source”). In so doing, any bacterial spores present in or on the object will be inactivated.

In one embodiment, food can be decontaminated using methods of the present invention. “Food” includes, but is not limited to, animal-derived products (such as meat, fish, milk, cheese and eggs), plants (such as vegetables, grains, seeds, and oils), plant-derived products, and fungus/fungus-derived products (such as mushrooms, tofu, yeast and yeast-products). The food to be decontaminated can be for consumption by humans or other animals.

In another embodiment, the objects and substances that can be decontaminated using methods of the present invention include, but are not limited to, animal tissues for transplantation or grafting, products made from human or animal organs or tissues, serum proteins (such as albumin and immunoglobulin), extracellular matrix proteins, gelatin, hormones, bone meal, nutritional supplements, and additionally any material that can be found in a human or animal that is susceptible to infection or that may carry or transmit infection.

In another embodiment “biological fluids” can be decontaminated using methods of the present invention. Biological fluids include, but are by no means limited to, cerebrospinal fluid, blood, blood products, milk, and semen, and also includes culture medium used for the culture of cells or for the production of recombinant proteins. The term “blood product” includes the red blood cells, white blood cells, serum or plasma separated from the blood. A further aspect of the invention is the use of the claimed methods to treat blood and blood products prior to transfer to a recipient.

In another embodiment, the objects and substances that can be decontaminated using the methods of the present invention are medical instruments, such as catheters, cannulas, dialysis or transfusion devices, shunts, stents, sutures, scissors, needles, stylets, devices for accessing the interior of the body, implantable ports, blades, scalpels. The term “medical instrument” is intended to encompass any type of device or apparatus that is used to contact the interior or exterior of a patient and also includes dental instruments. The term also encompasses any device or tool used in the preparation or manufacture, or otherwise comes into contact with, a biological tissue.

In another embodiment, the objects and substances that can be decontaminated using methods of the present invention are “surfaces.” Surfaces include walls, floors, furniture, any object made of a solid material (such as materials made of wood, metal or plastic), hospital surfaces (such as operating tables) laboratory work surfaces, and food preparation surfaces.

In another embodiment, the objects and substances that can be decontaminated using methods of the present invention include clothing, for example clothing worn by rescue workers, members of the emergency services, members of the military, hospital workers and any clothing suspected of having been contaminated with bacterial spores.

In another embodiment, the objects and substances that can be decontaminated using methods of the present invention include machinery or equipment (such as hospital machinery, military machinery, industrial machinery and mail sorting equipment) and vehicles.

In another embodiment water and air supplies can decontaminated using methods of the present invention. This includes the air and water itself in addition to systems used to deliver air and water such as water tanks, pipes, ventilation ducts and heating/air-conditioning systems.

Bacterial spores to be inactivated in this way can be those of any bacterial species known in the art to produce spores, including those previously described herein.

C. Photosensitizers

Particular photosensitizers can be selected for use according to their: 1) efficacy in delivery, 2) wavelength of absorbance, 3) excitatory wavelength, and/or 4) safety.

In one embodiment the photosensitizers used are phenothiaziniums. In specific embodiments the phenothiaziniums include toluidine blue derivatives, toluidine blue O (TBO), methylene blue (MB), new methylene blue N (NMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, neutral red, phenothiazinium, 5-ethylamino-9-diethylaminobenzo[a]phenothiazinium chloride, phenoselenazinium, phenotellurazinium, 5-ethylamino-9-diethylaminobenzo[a]phenoselenazinium chloride, thiopyronine, and thionine.

Phenothiaziniums, when irradiated with visible light, cause the conversion of molecular oxygen to “reactive species” such as singlet oxygen and oxygen radicals. Importantly, Phenothiazinium dyes are known to be safe for use in medical applications. For example, the Phenothiazinium dyes Methylene blue (MB), toluidine blue (TB), and their derivatives have been used therapeutically as antidotes to carbon monoxide poisoning and in long-term therapy of diseases. Compositions containing Phenothiazinium dyes can be provided topically, orally or intravenously in high doses without any toxic effects. Because of their known low toxicity and their accepted use in medical practice, as well as their high photoactive potential, Phenothiazinium dyes are ideal for use in accordance with the present invention.

In another embodiment the photosensizers used are phenodiazinium dyes. In a specific embodiment the phenodiazinium dye is safranine O.

In another embodiment the photosensizers used are phenooxazinium dyes.

Photosensitizers for use with methods of the invention are well-known in the art, and methods for their synthesis and use are described in, for example, Patent Application Nos. US20040147508, US20030180224, GB0413910, EP1392666, GB0329809, GB0327672, NZ0529682, NO20035327, GB0324425, NZ0525420, GB0314374, NO20031310, WO0224226, NO20031310, WO02096896, CA2448303, GB224407, WO0224226, WO0224226 and CA2423252, and U.S. Pat. Nos. 5,952,329, 6,624,187, 6,4656,44, 6,140,500 and 5,371,081, the contents each of which are expressly incorporated herein by reference.

Photosensitizer compositions of the present invention comprise an “effective amount” of the photosensitizer. An “effective amount” is an amount that is sufficient to inactivate the bacterial spores following irradiation with a light source. Amounts can be readily determined by one skilled in the art by, for example, performing assays for spore viability following irradiation. Many such assays are known in the art and any of these can be used. For example, one can determine the whether spores have been inactivated by obtaining sample or aliquots of the bacterial spore source during or following irradiation and determining the amount of “colony-forming units” present in that sample or aliquot. For example, the number of “colony forming units” in a sample can be determined as taught by Jett et al. (1997) by performing serial 10-fold dilutions in PBS, streaking the diluted samples on agar plates, incubating the agar plates at 37° C. overnight, and counting the number of colonies formed following incubation.

The effective amount will vary depending on factors such as (1) the photosensitive dye used, (2) the pH of the photosensitive dye composition, (3) the tissue type/site to which the photosensitive dye composition is to be delivered, (4) the amount or concentration of bacterial spores which might be present, and (5) the condition of the individual. It is well within the level of skill in the art to vary the amounts and choice of photosensitizer to accommodate one or more of these parameters.

It is envisaged that in some situations, the effective amount will be determined by a physician or a member of the emergency services on a case-by-case basis. In other situations, a pre-determined amount will be administered, either by a doctor, other medical worker, or by the contaminated individual themselves. The effective amount may be administered in one or more doses. Administrations can be conducted as frequently as is needed until the desired outcome, in this case inactivation of bacterial spores, is achieved.

A photosensitizer composition according to the invention will contain a suitable concentration of a photosensitizer and may also comprise certain other components. In some embodiments photosensitizers of the present invention are formulated with pharmaceutically acceptable carriers or excipients, such as water, saline, aqueous dextrose, glycerol, or ethanol, and may also contain auxiliary substances such as wetting or emulsifying agents, and pH buffering agents.

A photosensitizer composition may also contain complexing agents such as antibodies, enzymes, peptides, chemical species or binding molecules. These complexing agents may be used to stabilize or carry the photosensitize, or improve its ability to penetrate the substance or object being decontaminated, while not adversely affecting its phototoxic properties.

Additionally the photosensitizer composition of the present invention can contain additional medicinal or pharmaceutical agents. For example, in one embodiment the photosensitizer compositions of the present invention can additionally contain an antibiotic, a sporicidal agent, a disinfecting agent, or an agent useful in promoting wound healing. In an alternative embodiment, the photosensitizer compositions of the present invention can be co-administered with separate compositions containing antibiotics, sporicides, disinfectants, or agents useful in promoting wound healing.

An appropriate photosensitizer composition can be supplied in various forms and delivered in a variety of ways depending on the specific application. Standard texts, such as Remington: The Science and Practice of Pharmacy, 17^{th} edition, Mack Publishing Company, incorporated herein by reference, can be consulted to prepare suitable compositions and formulations for administration, without undue experimentation.

As for methods of administering photosensitizer compositions, mention is made of U.S. Pat. Nos. 5,952,329, 5,807,881, 5,798,349, 5,776,966, 5,789,433, 5,736,563, and 5,484,803, which can be consulted and employed in the practice of the invention.

Compositions of the present invention are administered by a mode appropriate for the form of the composition and the tissue/site to be treated. Compositions can be supplied in solid, semi-solid or liquid forms, including tablets, capsules, powders, liquids, lotions, creams, suspensions, spays and aerosols.

In one embodiment, the photosensitizer compositions are administered topically to the skin, or in particular to cuts, abrasions or other wounds in the skin. In this case, suitable forms for administration of the photosensitizer composition include creams, lotions, washes, and sprays. Other routes of topical administration may include application to the hair or eyes. In the case of application to the eyes, a bathing solution or eye drops are a preferred form of delivery.

In one embodiment, the photosensitizer compositions of the present invention comprise a simple aqueous solution containing an effective amount of the desired photosensitizer in sterile water, phosphate buffered saline, or some other aqueous solvent. Additionally such aqueous solutions may also contain pH buffering agents and preservatives and antimicrobial agents. Typically the amount of the photosensitizer present in such an aqueous solution formulation is in the range of about 0.0001% to about 50% weight/volume, or the photosensitizer may be present at concentrations ranging from about 0.1 μM to about 100 mM.

Such aqueous solution formulations are well suited to applications where bathing solutions, such as soaks or eye drops, or sprays are required. The aqueous solution photosensitizer compositions of the present invention can be administered to a specific site on a living animal or may be used to bathe or douse the whole animal. For example, in one embodiment the compositions of the present invention may be animal or human “dips”.

Thus, in one embodiment an aqueous solution containing the desired photosensitizer is used to soak or spray an affected part of the body, such as, for example, the eyes, and then either at the same time or after bathing, the affected part of the body is irradiated with an effective source of light. As used herein “treatment” refers to the application of the photosensizer composition and the irradiation of the photosensitizer composition with an effective light source. Treatment may be performed only once, or may be repeated as desired until the bacterial spores are inactivated. For example, successive treatments at hourly intervals may be used. Alternatively, treatments may be performed twice daily, or as directed by a physician.

In other embodiments, the photosensitizer compositions can be applied topically in the form of creams, lotions, ointments and the like. Many formulations of suitable “base” creams and lotions for topical application are known in the art, and any such formulation can be used. By “base” is meant the formulation of the composition without the actual active substance. For example, in the case of an antibiotic cream, the “base” is all of the components of the cream other than the antibiotic. An effective amount of the chosen photosensitizer can be added to the “base” cream and lotion formulations as taught by U.S. Pat. Nos. 6,621,574, 5,874,098, 5,698,589, 5,153,230 and 6,607,753. The chosen photosensitizer can be mixed with any known “base” cream, ointment or lotion known in the art to be safe for topical application. In some embodiments, other active agents may be added to the photosensitizer composition, such as antibiotics or sporicidal agents. In other embodiments, the chosen photosensitizer can mixed with a premade composition that already contains one or more active ingredients such as an antibiotic or sporicidal agent. It is envisaged that the final concentration of the photosensitizer in the cream, lotion or ointment will be between about 0.0001% and about 50% of the final composition, depending upon factors such as the specific photosensitizer used.

Suitable compositions for the “base” of the creams, lotions, and ointments of the present invention comprise a solvent (such as water or alcohol), and an emollient (such as a hydrocarbon oil, wax, silicone oil, vegetable, animal or marine fat or oil, glyceride derivative, fatty acid or fatty acid ester, alcohol or alcohol ether, lecithin, lanolin and derivatives, polyhydric alcohol or ester, wax ester, sterol, phospholipid and the like), and generally also contain an emulsifier (nonionic, cationic or anionic), although some emollients inherently possess emulsifying properties and thus in these situations an additional emulsifier is not necessary. These “base” ingredients can be formulated into either a cream, a lotion, a gel, or a solid stick by utilization of different proportions of the ingredients and/or by inclusion of thickening agents such as gums, hydroxypropylmethylcellulose, or other forms of hydrophilic colloids.

In one embodiment, such photosensitizer-containing creams, ointments and lotions are applied topically to the skin, mucous membranes (such as the oral cavity) or hair and then irradiated with the effective light source. Such treatments may be performed only once, or as frequently as desired until the bacterial spores are inactivated. For example, successive cream treatments at hourly intervals by be used. Alternatively, treatment may be performed twice daily or as directed by a physician.

An alternative means of treatment is to produce photosensitizer compositions in dry powdered form that can be inhaled. Where delivery by inhalation is desired, as much as possible of the photosensitizer powder of the present invention should consist of particles having a diameter of less than about 10 microns, for example about 0.01 to about 10 microns or about 0.1 to about 6 microns, for example about 0.1 to about 5 microns, or agglomerates of said particles. Preferably at least 50% of the powder consists of particles within the desired size range. These powders need not contain other ingredients. However compositions containing the photosensitizer powders of the present invention may also include other pharmaceutically acceptable additives such as pharmaceutically acceptable adjuvents, diluents and carriers. Carriers are preferably hydrophilic such as lactose monohydrate. Other suitable carriers include glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, and betaine.

Administration to the respiratory tract may be effected for example using a dry powder inhaler or a pressurised aerosol inhaler. Suitable dry powder inhalers include dose inhalers, for example the single dose inhaler known by the trade mark Monohaler™ and multi-dose inhalers, for example a multi-dose, breath-actuated dry powder inhaler such as the inhaler known by the trade mark Turbuhaler™.

In other embodiments, the photosensitizer compositions of the present invention are formulated for delivery by injection. In one embodiment a sterile solution the desired photosensitizer in an aqueous solvent (e.g. phosphate buffered saline) is administered be injection intradermally, subcutaneously, intramuscularly or, intravenously.

In other embodiments, compositions for injection also preferably include conventional pharmaceutically acceptable carriers and excipients which are known to those of skill in the art. Many different “base” formulations are known in the art to be suitable for preparation and delivery of active agents by injection, and any of these can be used. For example, suitable injectable “base” compositions are taught by U.S. Pat. No. 6,326,406.

Injectable photosensitizer compositions can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the injectable photosensitizer compositions to be administered may also contain minor amounts of non-toxic auxiliary substances such as wetting or emulsifying agents, pH buffering agents and the like, such as for example, sodium acetate, sorbitan monolaurate, triethanolamine oleate.

For example a formulation comprising a sterile solution of the desired photosensitizer at a concentration of about 1 μM to about 100 mM in physiological saline solution is injected intradermally, subcutaneously, intramuscularly, or intravenously. Treatment” is then completed by irradiating the affected individual, or a specific site on that individual such as the injection site, with an effective light source, either at the time of, or following, the injection of the photosensitizer composition. In one embodiment the photosensitizer composition is injected in the vicinity of a region of the body that is believed to be contaminated with bacterial spores, such as a scratch, abrasions, cut or other wound in the skin. In other embodiments the photosensitizer composition may be delivered systemically, for example, by intravenous injection.

Injections and treatments may be performed only once, or as frequently as desired until the bacterial spores are inactivated. For example, successive treatments at hourly intervals may be used. Alternatively, treatment may be performed twice daily or as directed by a physician.

Another suitable method for administration of the photosensitizer compositions of the present invention is to implant a slow-release or sustained-release system, such that a constant level of dosage of the photosensitizer composition maintained. See, e.g., U.S. Pat. No. 3,710,795, which is incorporated herein by reference. Photosensitizer compositions may also be administered by transdermal patch (e.g., iontophoretic transfer) for local or systemic application. In both cases, the site of the implant or patch is irradiated with an effective light source to complete the treatment. Any of the above compositions can be pre-formulated in the desired form or can also be supplied as liquid solutions, suspensions, or emulsions, to be diluted prior to use, and as solids forms suitable for dissolution or suspension in liquid prior to use.

In a specific embodiment, the photosensitizer compositions are applied to mucous membranes of the respiratory tract, for example by oral, intranasal or intrapulmonary delivery. In mucosal application, a preferred composition is one that provides a solid, powder, or liquid aerosol when used with an appropriate aerosolizer device.

In the case of compositions for application to mucosal membranes, it is desirable for the compositions to have an isotonicity compatible with that of the mucosal secretions. The isotonicity of the composition may be adjusted accordingly using sodium chloride, or other pharmaceutically acceptable agents such as dextrose, boric acid, sodium tartrate, propylene glycol or other inorganic or organic solute. Sodium chloride is preferred.

In some situations the composition for application to mucosal membranes may be a simple aqueous solution containing an effective amount of the desired photosensitizer in sterile water, phosphate buffered saline, or some other aqueous solvent. Alternatively, the viscosity of compositions for application to mucosal membranes may be maintained at any desired level by using a therapeutically acceptable thickening agent. Methyl cellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Specific compositions for mucosal applications will also contain a humectant to inhibit drying of the mucous membrane and to prevent irritation. Any of a variety of therapeutically acceptable humectants can be employed including, for example sorbitonl propylene glycol or glycerol. As with the thickeners, the concentration will vary with the selected agent, although the presence of absence of these agents, or their concentration is not an essential feature of the invention.

For the inactivation of bacterial spores in or on the mucosal membranes of living animals, it is intended that aqueous solutions (with or without thickeners) are applied to the membranes in liquid droplet form, or in spray form where it produces a “bathing mist”. Alternatively, liquid compositions may be inhaled as aerosol sprays either via mouth or nose.

If desired, enhanced absorption across mucosal membranes can be accomplished by employing a therapeutically acceptable surfactant. Typically useful surfactants for these therapeutic compositions include polyoxyethylene derivatives of fatty acid partial esters of sorbitol anhydrides such as Tween 80, Polyoxyl 40 Stearate, Polyoxyethylene 50 Stearate and Octoxynol. The usual concentration is from 1% to 10% based on the total weight.

Treatment of the mucosal membranes, using any of the above compositions, is completed by irradiating the photosensitizer composition on the mucosal membranes with an effective source of light. Where the mucous membranes are easily accessible, any desired light source (such as natural sunlight, lamps, lasers, LEDs or fiber optic devices my be used. For treatment of less accessible sites such as the nasal cavity and lungs, a fiber optic device or small other small flexible light source, should be used.

A therapeutically acceptable preservative is generally employed to increase the shelf life of the compositions. Such preservatives can be used with all of the compositions of the present invention. Benzyl alcohol is suitable, although a variety of preservatives including, for example, parabens, thimerosal, chlorobutanol, or benzalkonium chloride may also be employed. A suitable concentration of the preservative will be from about 0.02% to about 2% based on the total weight, although there may be appreciable variation depending upon the agent selected.

For use in the inactivation of bacterial spores in or on inanimate substances and objects, photosensitizers of the present invention can be administered in a “photosensitizer composition” that contains extra components in addition to the photosensitive dye. For example, the photosensitizer compositions of the present invention can additionally contain cleansing agents, detergents, surfactants, astringents, abrasives, boric acid, salts of boric acid, citric acid, sodium bicarbonate, potassium bicarbonate, zinc sulfate, bacteriocides, sporicides, or protein denaturing agents. Alternatively, photosensitizer compositions of the present invention can be used in conjunction with separate decontaminating agents.

In other embodiments for decontaminating inanimate substances and objects, the photosensitizer compositions can be used in conjunction with other means of treatment of contaminated material, such as irradiation with U.V. or gamma rays, heat treatment, autoclaving, or filtration. In addition, photosensitizers can be added to any suitable liquid formulations known in the art to be useful for disinfecting or cleaning products. For example, the desired photosensitizer may be added to known liquid disinfectant and cleaning solutions such as those taught in U.S. Pat. Nos. 6,583,176, 6,530,384, and 6,309,470 at concentrations ranging from 1 μM to 1M.

In one embodiment, the photosensitizer compositions are applied to a specific part of an object to be decontaminated, such as an area that has been splashed with a suspension of bacterial spores, or onto which dry bacterial spores are believed to be located. In another embodiment, the photosensitizer compositions are applied to the entire object or can be used to soak or wash large amounts or volumes of a substance. Decontamination is effected by irradiating the substance or object to which the photosensitizer composistion has been applied, with an effective source of light.

In certain embodiments, the photosensitizer compositions of the present invention can be used to decontaminate biological fluids, for example, to decontaminate blood prior to its use in transfusion. Photosensitizers can be directly added to biological fluid, such as blood, without the need for removal prior to administration of the biological fluid to a patient. Following sustained irradiation, the photosensitizers become photobleached and are thus inactivated. This means that after the blood has been “treated” to inactivate any bacterial spores, the photosensitizer itself will become inactive and therefore biologically inert.

Thus, in one embodiment, a desired photosensitizer is added to a blood sample, which is then irradiated with an effective light source such that any bacterial spores in the blood sample are inactivated. Photosensitizers may be added directly to hospital blood bags, and the bags can then be irradiated directly. Any other means for treating blood samples with photosensitizers that are known in the art, such as those taught in U.S. Pat. Nos. 5,955,256 and 6,277,337, can be used.

Similarly, any other fluid, such as drinking water, can also be decontaminated in this way using the methods of the present invention. U.S. Pat. No. 6,277,337 teaches suitable methods and apparatuses that can be used for the treatment of fluids, such as water with photosensitizers. The methods taught in this U.S. patent can be applied to the methods of the present invention.

D. Light Sources

An effective source of light is one that is sufficient to activate a particular photosensitizer. Different photosensitizers require different ranges of wavelength, light dosage (fluence), intensity (fluence rate) and time of irradiation for photo-activation. These factors are known for all currently available photosensitizers and this information is readily obtainable, such as from product guidelines that are supplied with commercially available photosensitizers. Thus, determining what is an “effective source of light” for a given photosensitizer is well within ordinary skill in the art and requires no inventive effort.

For photoactivation, the wavelength of light is matched to the electronic absorption spectrum of the photosensitizer so that the photosensitizer absorbs photons and the desired photochemistry can occur. The wavelength of activating light should be tailored to the absorption band of particular photosensitizer. For use in decontamination of animals, the range of activating light is typically between about 400 to about 900 nm. Some biological molecules, in particular hemoglobin, strongly absorb light below 600 mm and therefore capture the incoming photons (Parrish et al., (1978) Optical properties of the skin and eyes. New York, N.Y.: Plenum). Activation in this range may impair penetration of the activating light through the tissue. Alternatively, activation at greater than 900 nm may not be sufficient to produce ¹O₂, the activated state of oxygen which, without wishing to necessarily be bound by any one theory, is advantageous for successful inactivation of bacterial spores. In addition, water begins to absorb at wavelengths greater than about 900 mm.

In specific embodiments, the activating light is provided at a wavelength of greater than about 400, 500, 600 or 700 nm, or in a range from about 450 m to about 750 m.

The effective penetration depth, δ_{eff}, of a given wavelength of light is a function of the optical properties of the material being irradiated, such as absorption and scatter. For example, the fluence (light dose) in a tissue is related to the depth, d, as: e^{-d}/δ_{eff}. Typically, the effective penetration depth is about 2 to about 3 mm at 630 nm and increases to about 5 to about 6 nm at longer wavelengths (700-800 nm) (Svaasand and Ellingsen, 1983). In general, photosensitizers with longer absorbing wavelengths and higher molar absorption coefficients at these wavelengths are more effective photosensitizers.

The effective light dosage will vary depending on various factors, including the amount of the photosensitizer administered, the wavelength of the photoactivating light, the intensity of the photoactivating light, and the duration of irradiation by the photoactivating light. Thus, the light dose can be adjusted to an effective dose by adjusting one or more of these factors. In general the total fluence applied should be in the range of about 10 to about 1000 J/cm². The determination of suitable wavelength, light intensity, and duration of irradiation is within ordinary skill in the art.

In embodiments where the photosensitizer is methylene blue (MB), it is preferred that that the irradiating light has a wavelength of about 660 m and a fluence of up to about 1000 J/cm².

In embodiments where the photosensitizer is New Methylene Blue (NMB) it is preferred that that the irradiating light has a wavelength of about 635 nm and a fluence of up to about 1000 J/cm².

In embodiments where the photosensitizer is 1,9-Dimethylmethylene Blue Chloride (DMMB) it is preferred that that the irradiating light has a wavelength of about 660 nm and a fluence of up to about 1000 J/cm².

In embodiments where the photosensitizer is methylene green it is preferred that that the irradiating light has a wavelength of about 660 nm and a fluence of up to about 1000 J/cm².

In embodiments where the photosensitizer is methylene violet Bernthsen it is preferred that that the irradiating light has a wavelength of about 600 nm and a fluence of up to about 1000 J/cm².

In embodiments where the photosensitizer is methylene violet 3RAX it is preferred that that the irradiating light has a wavelength of about 560 nm and a fluence of up to about 1000 J/cm².

In embodiments where the photosensitizer is malachite green it is preferred that that the irradiating light has a wavelength of about 610 mm and a fluence of up to about 1000 J/cm².

In embodiments where the photosensitizer is either toluidine blue (TB) or toluidine blue O (TBO) it is preferred that that the irradiating light has a wavelength of about 635 nm and a fluence of up to about 1000 J/cm².

In embodiments where the photosensitizer is either azure blue A or azure blue B it is preferred that that the irradiating light has a wavelength of about 620 nm and a fluence of up to about 1000 J/cm².

In embodiments where the photosensitizer is azure blue C it is preferred that that the irradiating light has a wavelength of about 600 nm and a fluence of up to about 1000 J/cm².

In embodiments where the photosensitizer is neutral red it is preferred that that the irradiating light has a wavelength of about 540 nm and a fluence of up to about 1000 J/cm².

In embodiments where the photosensitizer is thionine it is preferred that that the irradiating light has a wavelength of about 600-nm and a fluence of up to about 1000 J/cm².

The light for photoactivation can be produced and delivered by any suitable means known in the art. In one embodiment a strong light source such as a searchlight, lamp, light box, laser, light-emitting diode (LED) or optical fiber is used to irradiate the animal or object until the required fluence has been delivered.

In another embodiment natural sunlight is used as light source. Photosensitive dyes are, by definition, light sensitive. Thus, they are totally photobleached and/or degraded following long prolonged exposure to sunlight.

If natural sunlight is used it is preferred, although not essential, that a light meter is used to measure the light dose and dose rate in order that the object or animal is exposed to the sunlight for a sufficient period of time. In some circumstances, such as for decontamination in the field during combat, or for decontamination of large objects or large numbers of people, the use of natural sunlight may be particularly advantageous as it eliminates the need for large numbers of artificial light sources which may be in short supply and may be cumbersome and/or expensive. Furthermore, the use of natural sunlight as the light source is also desirable from an environmental point of view.

The present invention is additionally described by way of the following illustrative, non-limiting examples, which provide a better understanding of the present invention and its many advantages.

### EXAMPLES

### Example 1

### Bacillus Species Studied, Methods of Culture and PDI Methods

As access to B. anthracis is highly regulated, much of the research into Anthrax is now performed using B. cereus as a surrogate. B. cereus is very closely related to B. anthracis and a recent report suggests that from a genetic viewpoint they are the same species (Helgason et al., 2000). A similar argument is made regarding B. thuringiensis which is widely used as a biological insecticide. In fact, there is mention of the B. anthracis “cluster” that includes all B. anthracis strains (both pathogenic and non-pathogenic) together with numerous B. cereus and B. thuringiensis strains (Schuch et al., 2002). While B. cereus is most widely known as a cause of food-borne illness (Carlin et al., 2000), it not infrequently causes localized tissue infections in humans after gunshot wounds (Krause et al., 1996) or other trauma (Akesson et al., 1991; Krause et al., 1996) and the spores are thought to be equally resistant to sporicidal agents as are those of B. anthracis (Lensing & Oei, 1985).

The bacteria studied in the following examples were B. atrophaeus (ATCC 9372), B. cereus (ATCC14579), B. thuringiensis (ATCC 33740) and B. subtilis (ATCC 6051). Growing bacterial cells were cultivated in brain-heart infusion (BHI) broth at 37° C. Aliquots of the suspension (10⁸/mL) were stored at −80° C. and then used for the experiments.

For initial experiments spores of B. atrophaeus and B. cereus were purchased from SGM Biotech, Inc (Bozeman, Mont., USA). For subsequent experiments spores of all species were prepared in the laboratory using sporulation broth for B. atrophaeus and B. subtilis, or sporulation agar (Caipo et al., 2002; Nicholson & Setlow, 1990) for B. cereus and B. thuringiensis. The sporulation medium consisted of 16.0 g nutrient broth (Difco), 2.0 g KCl, 0.5 g MgSO₄; 17 g of agar. The pH of the medium was adjusted to 7, then autoclaved and cooled. After cooling 1 ml of 1M Ca₂(NO₃)₂, 1 ml of 0.1 M MnCl₂.4H₂O, 1 ml of 1 mM FeSO₄ and 2 ml 50% glucose were added. For spore purification the mixture of spores and cells was centrifuged at 1300 g for 20 min, washed with SX volume 1 M KCl/0.5 M NaCl, rinsed with sterile deionized water, then washed with 1 M NaCl, and rinsed with sterile deionized water again. Lysozyme (50 μg/mL) was added in the presence of buffer (5× volume Tris Cl, 0.05 M, pH 7 2), and incubated with constant stirring at 4° C. overnight. Lysozyme was removed by centrifuging 8 times (at 1300 g) and washing with sterile deionized water. Spores were frozen with 10% glycerol and stored until use. To avoid germination spores were used immediately after defrosting.

As photosensitizers Rose Bengal, Toluidine Blue O (TBO), Methylene Blue, New Methylene Blue N zinc chloride double salt (NMB), 1,9-Dimethylmethylene Blue Chloride (Sigma-Aldrich—DMMB), Azure A, Azure B, Azure C, methylene violet 3RAX, safranine O, and malachite green, were used. Stock solutions were prepared in water and stored at 4° C. in the dark before use. The concentrations of stock solution were 2 mM.

When Rose Bengal was used, irradiation was performed with an argon laser at 514 nm. A diode laser with wavelength 670 nm was used for experiments with Methylene Blue and 1,9-Dimethylmethylene Blue Chloride. Diode laser with wavelength 635 nm was used for Toluidine Blue O and New Methylene Blue N. For other dyes either a turnable argon ion pumped dye laser or a 514 nm argon ion laser was used.

Suspensions of spores or bacteria (10⁸/mL, 10⁷/mL, 10⁶/mL) were incubated with photosensitizers in the dark at room temperature. Incubation time was ranged from 1 min to 24 h and the photosensitizer concentrations varied form 10 μM to 1 mM. The cell suspensions were centrifuged at 20800 g and then washed several times with sterile PBS. The bacterial suspensions were placed on two well (concavities hanging drop) slides (Fisher Scientific) and irradiated with appropriate laser at room temperature. Fluences ranged from 0 to 300 J/cm². Fluence rates varied from 0 to 500 mW/cm². During irradiation aliquots of 20 μL were taken to determine the colony-forming units. The contents of the wells were mixed before sampling. The aliquots were serially diluted 10-fold in PBS to give dilutions of 10⁻¹-10⁻⁶ times the original concentrations and were streaked horizontally on square BHI agar plates as described by (Jett et al., 1997). Plates were incubated at 37° C. overnight.

Two types of control conditions were used: irradiation in the absence of photosensitizers and incubation with photosensitizers in the dark.

The data presented in the following Examples indicates that bacterial spores can be destroyed using a combination of photosensitive dyes and irradiation with light within the visible range.

### Example 2

### Effect of Toludine Blue on survival of B. cereus Spores

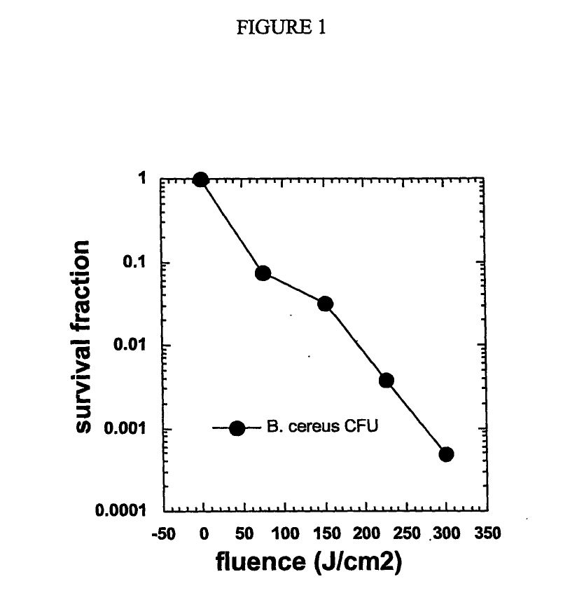

As shown in FIG. 1, when B. cereus spores were incubated with 100 μM TBO for 10 minutes and irradiated with 100 mW/cm² 635-nm light, greater than 99.9% of the spores were killed.

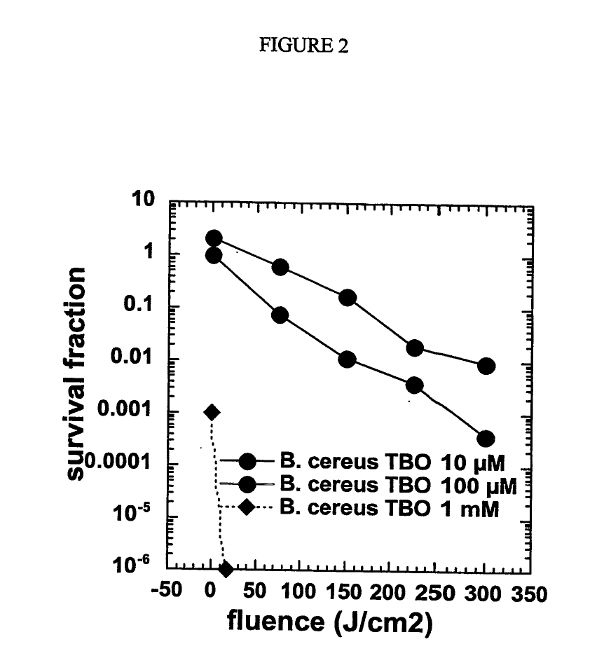

The data shown in FIG. 2 illustrate the effect of different concentrations of TBO. B. cereus spores were incubated with either 10 μM, 100 μM or 1 mM TBO for 10 minutes and irradiated with 100 mW/cm² 635-nm light. The killing of B. cereus spores was found to be improved, depending on both TBO concentration and light fluence. At the 1 mM dose, TBO exhibited significant dark toxicity to spores, and complete killing of spores at the first lowest light dose tested.

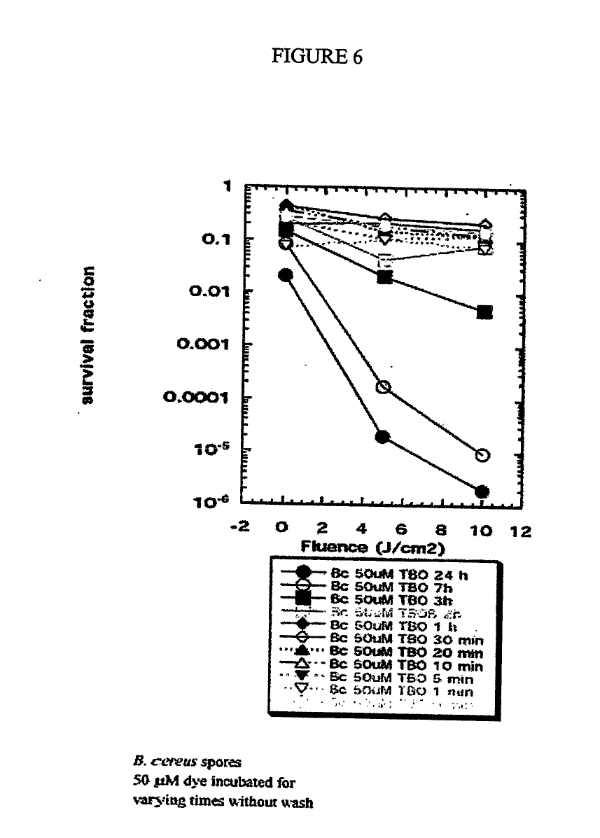

FIG. 6 illustrates the effect of varying incubation periods on the effectiveness of TBO in PDI. Spores were incubated in 50 μM TBO for various times ranging from 1 minute to 24 hours. Irradiation was either applied concurrently with photosensitizer incubation, or subsequent to photosensitizer incubation. Both methods worked well, with different methods being preferable for different dyes. It can be seen that the effectiveness of killing increases with increasing incubation time. Incubation periods of 3 hours or more appeared to be the most effective at this concentration of TBO.

### Example 3

### Comparison of the Effect of Toludine Blue in PDI with B. cereus, B. thuringiensis, B. subtilis and B. atrophaeus Spores

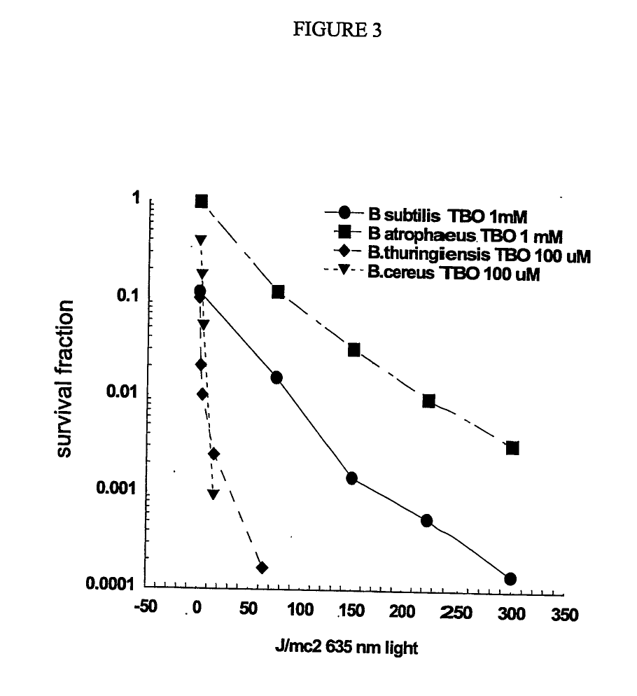

The data presented in FIG. 3 shows the effect of TBO on various different Bacillus species. B. cereus and B. thuringiensis were the most susecptible to PDI, requiring one tenth the amount of dye and one sixth the amount of light to produce more than 99.9% killing as compared to B. subtilis and B. athrophaeus.

### Example 4

### Effect of Spore Concentration of Survival of B. cereus Spores Following PDI

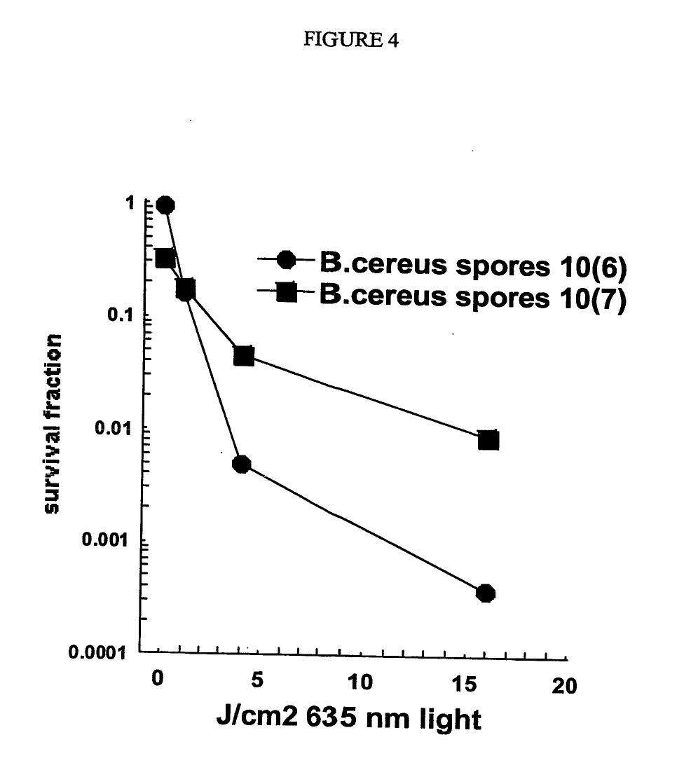

The data presented in FIG. 4 shows that B. cereus spores are more sensitive to PDI when they are diluted. In this case it was found that a tenfold dilution in the suspension of B. cereus spores (from 10⁷ spores/mL to 10⁶ spores/mL) resulted in an increase in amount of spore killing for a given fluence of light. This experiment was carried out with 100 μM TBO and a 30 minute incubation time.

### Example 5

### Photodynamic Sporicidal Activity of Methylene Blue, AzureA, AzureB, and Azure C

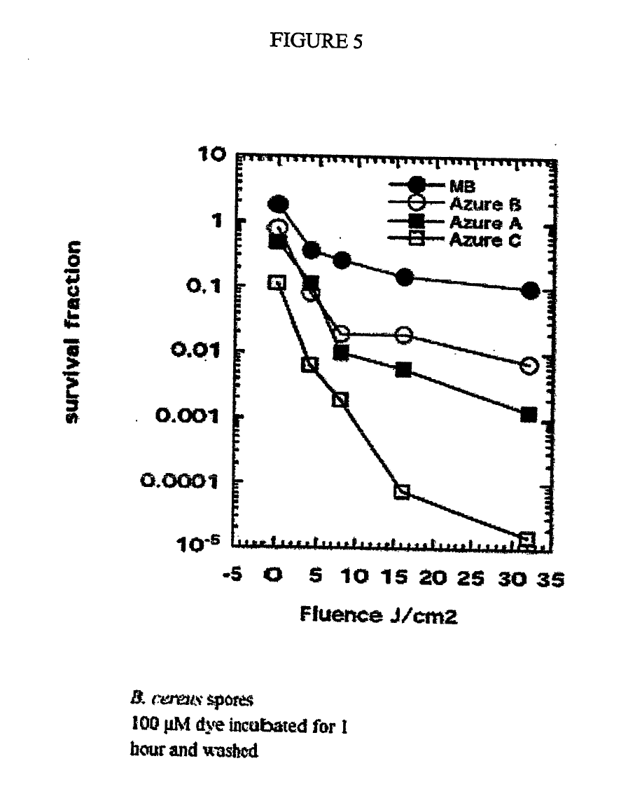

Various other photosensitizing dyes were tested for their ability to mediate photodynamic killing of Bacillus spores. The dyes tested include methylene blue, AzureA, AzureB, and Azure C. As can be seen from FIG. 5, all of these dyes were found to be effective in killing Bacillus spores by PDI. Of the dyes for which data is shown in FIG. 5, Azure C was the most potent, followed by Azure B, Azure B and methylene blue.

Based on this data it was determined that dyes comprising phenothiazinium, phenooxazinium, phenodiazinium or phenoselenazinium salts should be effective photodynamic sporicidal agents. Such dyes include methylene blue derivatives (such as dimethylmethylene blue—DMMB), methylene green, methylene violet Bernthsen, methyleneviolet 3RAX, safranine O, and neutral red. This hypothesis was subsequently tested and found to be correct.

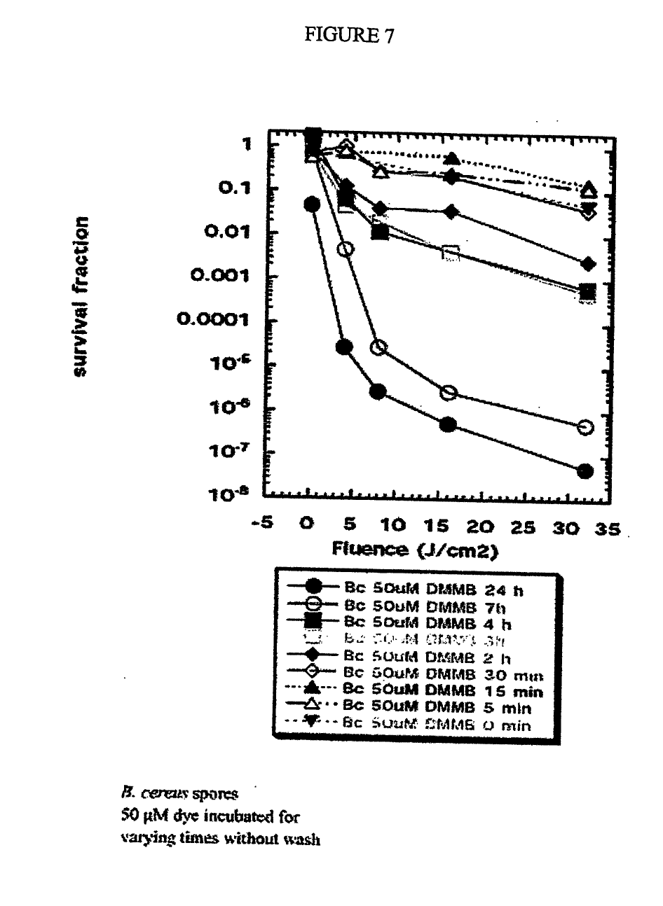

Dimethylmethylene Blue (DMMB) was found to be effective in killing B. cereus spores in PDI. FIG. 7 shows the effect of varying incubation periods on the effectiveness of DMMB. Spores were incubated in 50 μM DMMB for times ranging from 1 minute to 24 hours. It can be seen that the effectiveness of killing increases with increasing incubation time.

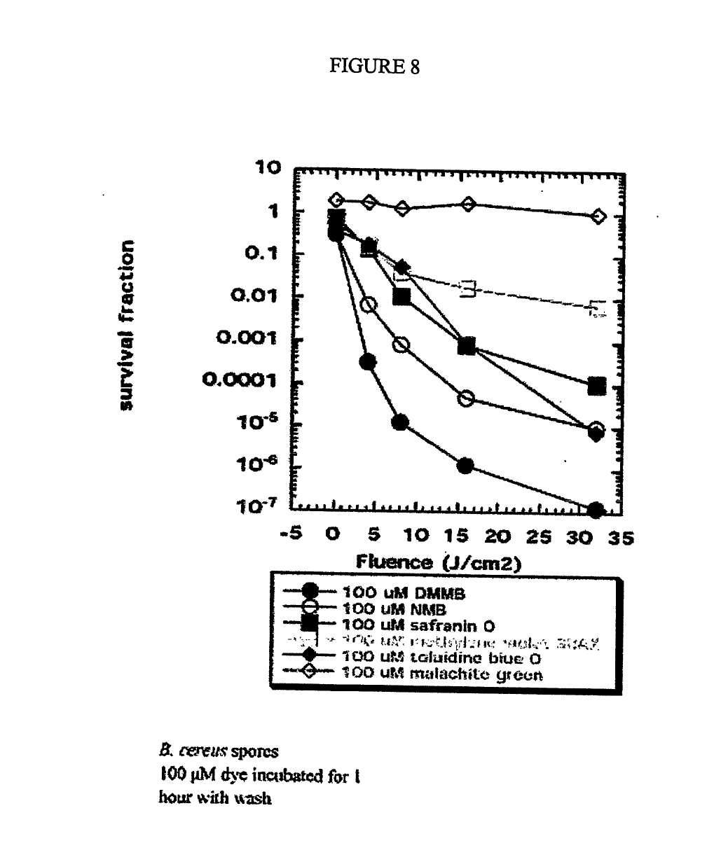

Other dyes that were found to be effective in killing B. cereus spores in PDI included “new methylene blue” (NMB), safranin O, methylene violet 3RAX and malachite green, as can be seen from FIG. 8.

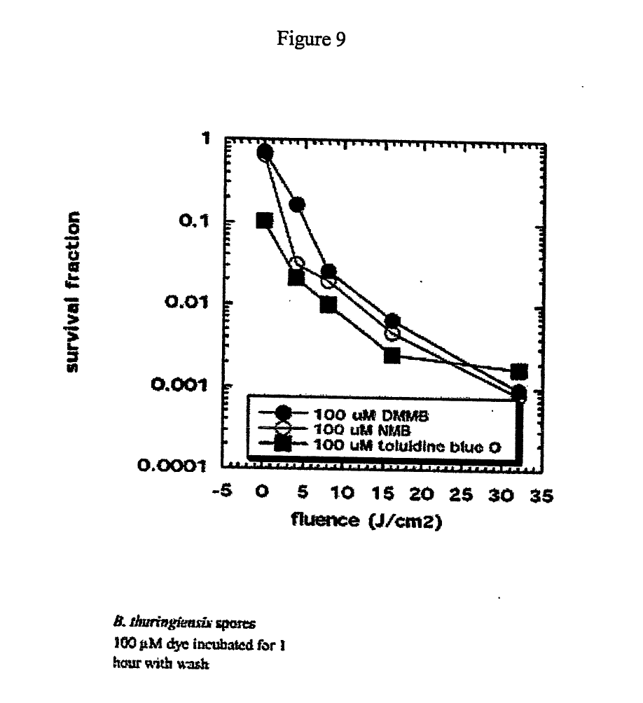

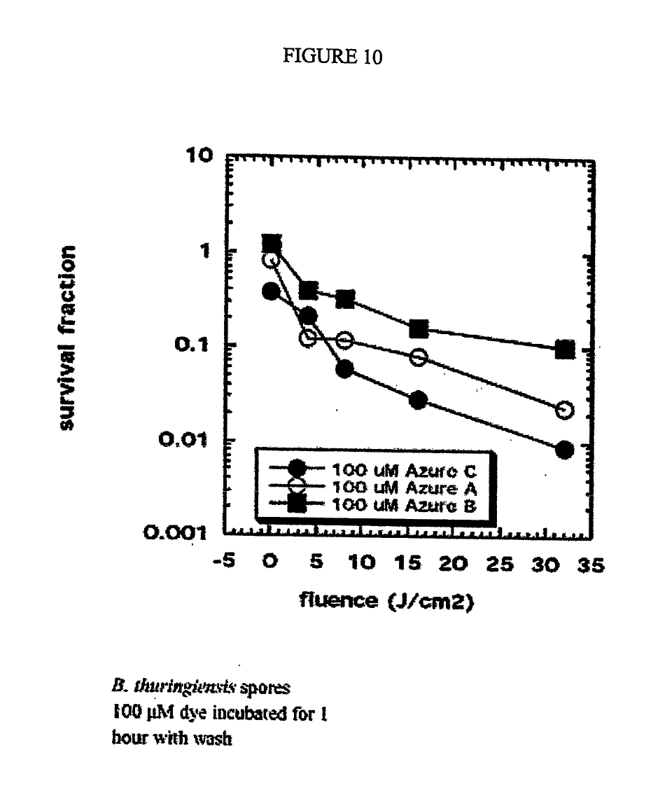

DMMB, NMB and TBO (see FIG. 9) and Azure A, AzureB, and Azure C (see FIG. 10) were also found to be effective in killing B. thuringiensis spores.

### Example 6

### Photoinactivation of Spores with Isosteric Dyes

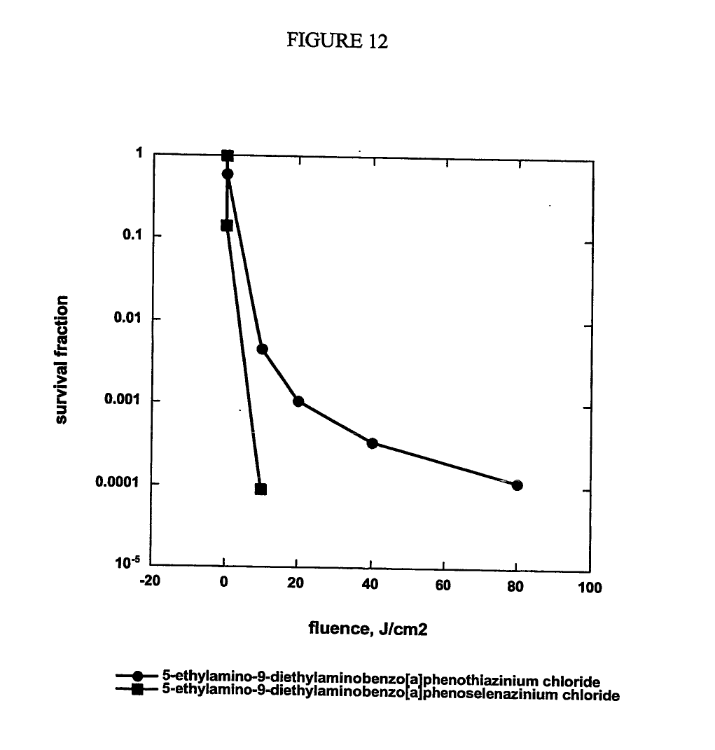

Photoinactivation of B. cereus spores with two isosteric dyes was also performed. B. cereus spores (10(6)/mL) were incubated with S-ethylamino-9diethylaminobenzo[a]phenothiazinium chloride (100 μM) and 5-ethylamino-9diethylaminobenzo[a]phenoselenazinium chloride (100 M) for 1 hour at 22° C., followed by irradiation with 240 mW/cm² 665-nm light. FIG. 12 illustrates the heavy atom effect in which substituting selenium for sulfur enhances triplet lifetime and singlet oxygen quantum yield.

### Example 7

### Photoinactivation of Spores vs Vegetative Cells

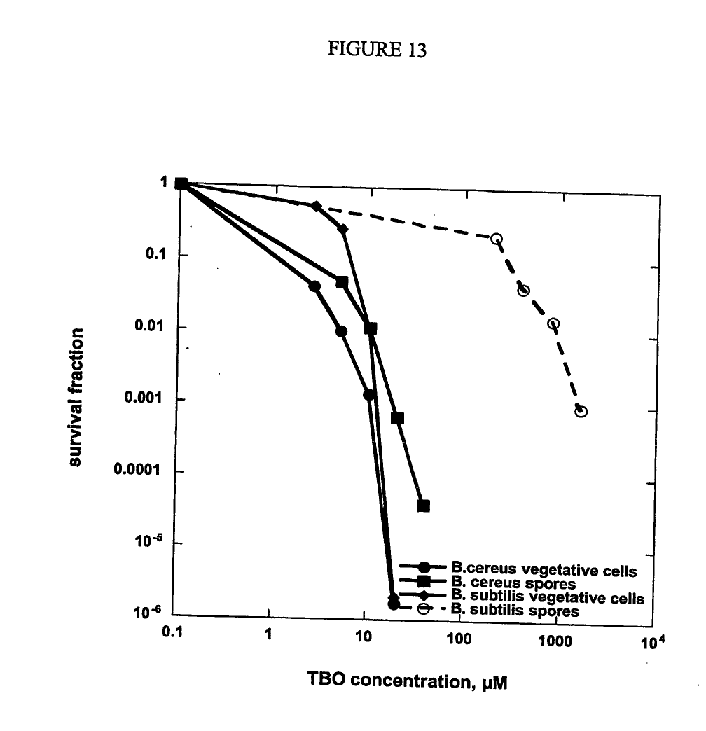

A comparison of photoinactivation of spores and corresponding vegetative cells from two Bacillus species was performed. B. cereus and B. cereus spores and cells (10(7)/mL) were incubated with toluidine blue for 3 hours at 37° C., followed by irradiation with 100 mW/cm² 635-nm light. FIG. 13 illustrates that the B. subtilis and B. cereus vegetative cells are sensitive to PDI. The sensitivity of the corresponding spores differs both from each other and from the vegetative cells.

Having thus described in detail preferred embodiments of the present invention, it is to be understood that the invention defined by the above description, examples and claims is not to be limited to the particular details set forth above, as many apparent variations thereof are possible without departing from the spirit or scope of the present invention.

### REFERENCES

Akesson, A., Hedstrom, S. A. & Ripa, T. (1991). Bacillus cereus: a significant pathogen in postoperative and post-traumatic wounds on orthopaedic wards. Scand J Infect Dis, 23: 71-7.
Athar, M., Mukhtar, H. & Bickers, D. R. (1988). Differential role of reactive oxygen intermediates in photofrin-I- and photofrin-II-mediated photoenhancement of lipid peroxidation in epidermal microsomal membranes. J Invest Dermatol, 90: 652-7.
Barr, H. (2000). Barrett's Esophagus: Treatment with 5-Aminolevulinic Acid Photodynamic Therapy. Gastrointest Endosc Clin N Am, 10: 421-437.
Bellin, J. S., Lutwick L. & Jonas, B. (1969). Effects of photodynamic action on E. coli. Arch Biochem Biophys, 132: 157-64.
Bertoloni, G., Rossi, F., Valduga, G., Jori, G. & van Lier, J. (1990). Photosensitizing activity of water- and lipid-soluble phthalocyanines on Escherichia coli. FEMS Microbiol. Lett., 59: 149-55.
Bezman, S. A., Burtis, P. A., Izod, T. P. & Thayer, M. A. (1978). Photodynamic inactivation of E. coli by rose bengal immobilized on polystyrene beads. Photochem Photobiol, 28: 325-9.
Bhatti, M., MacRobert, A., Meghji, S., Henderson, B. & Wilson, M. (1998). A study of the uptake of toluidine blue O by Porphyromonas gingivalis and the mechanism of lethal photosensitization. Photochem Photobiol, 68: 370-6.
Boehncke, W. H., Elshorst-Schmidt, T. & Kaufmann, R. (2000). Systemic photodynamic therapy is a safe and effective treatment for psoriasis. Arch Dermatol, 136: 271-2.
Booth, J. C. & Stern, H. (1972). Photodynamic inactivation of rubella virus. J Med Microbiol, 5: 515-28.
Boyle, R. W. & Dolphin, D. (1996). Structure and biodistribution relationships of photodynamic sensitizers. Photochem. Photobiol., 64: 469-485.
Brendel, M. (1973). Different photodynamic action of proflavine and methylene blue on bacteriophage. II. Mutation induction in extracellularly treated Serratiaphage kappa. Mol Gen Genet, 120: 171-80.
Bressler, N. M. & Bressler, S. B. (2000). Photodynamic therapy with verteporfin (Visudyne): impact on ophthalmology and visual sciences. Invest Ophthalmol Vis Sci, 41: 624-8.
Caipo, M. L., Duffy, S., Zhao, L. & Schaffner, D. W. (2002). Bacillus mdgaterium spore germination is influenced by inoculum size. Journal of applied microbiology, 92: 879-884.
Carlin, F., Girardin, H., Peck, M. W., Stringer, S. C., Barker, G. C., Martinez, A., Fernandez, A., Fernandez, P., Waites, W. M., Movahedi, S., van Leusden, F., Nauta, M., Moezelaar, R., Torre, M. D. & Litman, S. (2000). Research on factors allowing a risk assessment of spore-forming pathogenic bacteria in cooked chilled foods containing vegetables: a FAIR collaborative project. Int J Food Microbiol, 60: 117-35.
Chaudry, G. J., Moayeri, M., Liu, S. & Leppla, S. H. (2001). Quickening the pace of anthrax research: three advances point towards possible therapies. Trends Microbiol, 10: 58-62.
Cohn, G. E. & Tseng, H. Y. (1977). Photodynamic inactivation of yeast sensitized by eosin Y. Photochem Photobiol, 26: 465-74.
Cooney, J. J. & Krinsky, N. I. (1972). Photodynamic killing of Acholeplasma laidlawii. Photochem Photobiol, 16: 523-6.
Dahl, T. A., Midden, W. R. & Hartman, P. E. (1987). Pure singlet oxygen cytotoxicity for bacteria. Photochem Photobiol, 46: 345-52.
Dahl, T. A., Midden, W. R & Hartman, P. E. (1989). Comparison of killing of gram-negative and gram-positive bacteria by pure singlet oxygen. J Bacteriol, 171: 2188-94.
de Mol, N. J., Beijersbergen van Henegouwen, G. M., Mohn, G. R., Glickman, B. W. & van Kleef, P. M. (1981). On the involvement of singlet oxygen in mutation induction by 8-methoxypsoralen and UVA irradiation in Escherichia coli K-12. Mutat Res, 82: 23-30.
Dougherty, T. J., Gomer, C. J., Henderson, B. W., Jori, G., Kessel, D., Korbelik, M., Moan, J. & Peng, Q. (1998). Photodynamic therapy. J Natl Cancer Inst, 90: 889-905.
Driks, A. (2002). Maximum shields: the assembly and function of the bacterial spore coat. Trends in microbiology, 10: 251-254.
Dull, P. M., Wilson, K. E., Kournikakis, B., Whitney, E. A., Boulet, C. A., Ho, J. Y., Ogston, J., Spence, M. R., McKenzie, M. M., Phelan, M. A., Popovic, T. & Ashford, D. (2002). Bacillus anthracis aerosolization associated with a contaminated mail sorting machine. Emerg. Infect. Dis., 8: 1044-1047.
Friedberg, J. S., Tompkins, R. G., Rakestraw, S. L., Warren, S. W., Fischman, A. J. & Yarmush, M. L. (1991). Antibody-targeted photolysis. Bacteriocidal effects of Sn (IV) chlorin e6-dextran-monoclonal antibody conjugates. Ann N Y Acad Sci, 618: 383-93.
Gilligan, P. H. (2002). Therapeutic challenges posed by bacterial bioterrorism threats. Current opinion in microbiology, 5: 489-495.
Gomer, C. J. (1991). Preclinical examination of first and second generation photosensitizers used in photodynamic therapy. Photochem Photobiol, 54: 1093-107.
Gross, S., Brandis, A., Chen, L., Rosenbach-Belkin, V., Roehrs, S., Scherz, A. & Salomon, Y. (1997). Protein-A-mediated targeting of bacteriochlorophyll-IgG to Staphylococcus aureus: a model for enhanced site-specific photocytotoxicity. Photochem Photobiol, 66: 872-8.
Hamblin, M. R. & Newman, E. L. (1994). On the mechanism of the tumour-localising effect in photodynamic therapy. J Photochem Photobiol B, 23: 3-8.
Hamblin, M. R., Zahra, T., Contag, C. H., McManus, A. T. & Hasan, T. (2003). Optical monitoring and treatment of potentially lethal wound infections in vivo. The Journal of infectious diseases, 187: 1717-1726.
Harrison, A. P., Hafner, J. L. & O'Bryan, C. (1972). Ultraviolet-killing and photodynamic-killing in mutants of Escherichia coli. Mutat Res, 14: 447-9.
Hass, B. S. & Webb, R. B. (1979). Photodynamic effects of dyes on bacteria III. Mutagenesis by acridine orange and 500-nm monochromatic light in strains of Escherichia coli that differ in repair capability. Mutat Res, 60: 1-11.
Hass, B. S. & Webb, R. B. (1981). Photodynamic effects of dyes on bacteria. IV. Lethal effects of acridine orange and 460- or 500-nm monochromatic light in strains of Escherichia coli that differ in repair capability. Mutat Res, 81: 277-85.
Hausmann, W. (1908). Die sensibilisierende Wirkung tierscher Farbstoffe und ihne physiologische Bedeutung. Wien. Klin. Wochnschr., 21: 1527-1529.
Helgason, E., Okstad, O. A., Caugant, D. A., Johansen, H. A., Fouet, A., Mock, M., Hegna, I. & Kolsto. (2000). Bacillus anthracis, Bacillus cereus, and Bacillus thuringiensis-one species on the basis of genetic evidence. Appl Environ Microbiol, 66: 2627-30.
Henney, J. E. (2000). From the Food and Drug Administration. Jama, 283: 2779.
Hongcharu, W., Taylor, C. R., Chang, Y., Aghassi, D., Suthamjariya, K. & Anderson, R. R. (2000). Topical ALA-Photodynamic Therapy for the Treatment of Acne Vulgaris. J Invest Dermatol, 115: 183-192.
Houba-Herin, N., Calberg-Bacq, C. M., Piette, J. & Van de Vorst, A. (1982). Mechanisms for dye-mediated photodynamic action: singlet oxygen production, deoxyguanosine oxidation and phage inactivating efficiencies. Photochem Photobiol, 36: 297-306.
Imray, F. P. & MacPhee, D. G. (1973). The role of DNA polymerase I and the rec system in survival of bacteria and bacteriophages damaged by the photodynamic action of acridine orange. Mol Gen Genet, 123: 289-98.
Jacob, H. E. (1971). In vivo production of DNA single-strand breaks by photodynamic action. Photochem Photobiol, 14: 743-5.
Jacob, H. E., Fleck, W. & Strauss, D. (1977). [Photodynamic activity and repair inhibition of photodynamically produced DNA damages in proteus mirabilis by the anthracycline antibiotic violamycin BI]. Z Allg Mikrobiol, 17: 569-73.
Jacob, H. E. & Hamann, M. (1975). Photodynamic alterations of the cell envelope of Proteus mirabilis and their repair. Photochem Photobiol, 22: 237-41.
Jenkins, M. P., Buonaccorsi, G. A., Raphael, M., Nyamekye, I., McEwan, J. R., Bown, S. G. & Bishop, C. C. (1999). Clinical study of adjuvant photodynamic therapy to reduce restenosis following femoral angioplasty. Br J Surg, 86: 1258-63.
Jesionek, A. & von Tappenier, H. (1903). Zur behandlung der hautcarcinomit mit fluorescierenden stoffen. Muench. Med. Wochneshr., 47: 2042.
Jett, B. D., Hatter, K. L., Huycke, M. M. & Gilmore, M. S. (1997). Simplified agar plate method for quantifying viable bacteria. Biotechniques, 23: 648-50.
Kimmel, S. R., Mahoney, M. C. & Zimmerman, R. K. (2003). Vaccines and bioterrorism: smallpox and anthrax. J. Fam. Pract., 52: S56-61.
Krause, A., Freeman, R., Sisson, P. R & Murphy, O. M. (1996). Infection with Bacillus cereus after close-range gunshot injuries. J Trauma, 41: 546-8.
Kubin, A., Wierrani, F., Jindra, R. H., Loew, H. G., Grunberger, W., Ebermann, R. & Alth, G. (1999). Antagonistic effects of combination photosensitization by hypericin, meso-tetrahydroxyphenylchlorin (mTHPC) and photofrin II on Staphylococcus aureus. Drugs Exp Clin Res, 25: 13-21.
Lensing, H. H. & Oei, H. L. (1985). Investigations on the sporicidal and fungicidal activity of disinfectants. Zentralbl Bakteriol Mikrobiol Hyg [B], 181: 487-95.
Lu, X. M., Fischman, A. J., Stevens, E., Lee, T. T., Strong, L., Tompkins, R. G. & Yarmush, M. L. (1992). Sn-chlorin e6 antibacterial immunoconjugates. An in vitro and in vivo analysis. J Immunol Methods, 156: 85-99.
Lutwick, L. I. & Nierengarten, M. B. (2002). Vaccines for Category A bioterrorism diseases. Expert Opin. Biol. Ther., 2: 883-893.
Malik, Z., Ladan, H. & Nitzan, Y., (1992). Photodynamic inactivation of Gram-negative bacteria: problems and possible solutions. J. Photochem. Photobiol. B, 14: 262-6.
Malik, Z., Ladan, H., Nitzan, Y. & Ehrenberg, B. (1990). The bactericidal activity of a deuteroporphyrin-hemin mixture on gram-positive bacteria. A microbiological and spectroscopic study. J. Photochem. Photobiol. B, 6: 419-30.
Martin, J. P. & Logsdon, N. (1987). The role of oxygen radicals in dye-mediated photodynamic effects in Escherichia coli B. J Biol Chem, 262: 7213-9.
Merchat, M., Bertolini, G., Giacomini, P., Villanueva, A. & Jori, G. (1996a). Meso-substituted cationic porphyrins as efficient photosensitizers of gram-positive and gram-negative bacteria. J. Photochem. Photobiol. B, 32: 153-7.
Merchat, M., Spikes, J. D., Bertoloni, G. & Jori, G. (1996b). Studies on the mechanism of bacteria photosensitization by meso-substituted cationic porphyrins. J. Photochem. Photobiol. B, 35: 149-57.
Millson, C. E., Wilson, M., Macrobert, A. J., Bedwell, J. & Bown, S. G. (1996). The killing of Helicobacter pylori by low-power laser light in the presence of a photosensitiser. J. Med. Microbiol., 44: 245-52.
Minnock, A., Vernon, D. I., Schofield, J., Griffiths, J., Parish, J. H. & Brown, S. B. (1996). Photoinactivation of bacteria Use of a cationic water-soluble zinc phthalocyanine to photoinactivate both gram-negative and gram-positive bacteria. J. Photochem. Photobiol. B, 32: 159-64.
Minnock, A., Vernon, D. I., Schofield, J., Griffiths, J., Parish, J. H. & Brown, S. B. (2000). Mechanism of uptake of a cationic water-soluble pyridinium zinc phthalocyanine across the outer membrane of Escherichia coli. Antimicrob Agents Chemother, 44: 522-7.
Nicholson, W. L. & Galeano, B. (2003). UV resistance of Bacillus anthracis spores revisited: validation of Bacillus subtilis spores as UV surrogates for spores of B. anthracis Sterne. Applied and Environmental Microbiology, 69: 1327-1330.
Nicholson, W. L. & Setlow, P. (1990). Dramatic increase in negative superhelicity of plasmid DNA in the forespore compartment of sporulating cells of Bacillus subtilis. Journal of Bacteriology, 172: 7-14.
Nitzan, Y., Balzam-Sudakevitz, A. & Ashkenazi, H. (1998). Eradication of Acinetobacter baumannii by photosensitized agents in vitro. J Photochem Photobiol B, 42: 211-8.
Nitzan, Y., Gutterman, M., Malik, Z. & Ehrenberg, B. (1992). Inactivation of gram-negative bacteria by photosensitized porphyrins. Photochem. Photobiol., 55: 89-96.
Ochsner, M. (1997). Photophysical and photobiological processes in the photodynamic therapy of tumours. J Photochem Photobiol B, 39: 1-18.
Perlin, M., Mao, J. C., Otis, E. R., Shipkowitz, N. L. & Duff, R. G. (1987). Photodynamic inactivation of influenza and herpes viruses by hematoporphyrin. Antiviral Res, 7: 43-51.
Raab, C. (1900). Über die Wirkung fluoreszierender Stoffe auf Infusoria. Z. Biol., 39: 524-546.
Redmond, R. W. & Gamlin, J. N. (1999). A compilation of singlet oxygen yields from biologically relevant molecules. Photochem Photobiol, 70: 391-475.
Rockson, S. G., Lorenz, D. P., Cheong, W. F. & Woodburn, K. W. (2000). Photoangioplasty: An emerging clinical cardiovascular role for photodynamic therapy. Circulation, 102: 591-6.
Russell, A. D. (1990). Bacterial spores and chemical sporicidal agents. Clin Microbiol Rev, 3: 99-119.
Schafer, M., Schmitz, C., Facius, R., Homeck, G., Milow, B., Funken, K. H. & Ortner, J. (2000). Systematic study of parameters influencing the action of Rose Bengal with visible light on bacterial cells: comparison between the biological effect and singlet-oxygen production. Photochem Photobiol, 71: 514-23.
Schuch, R., Nelson, D. & Fischetti, V. A. (2002). A bacteriolytic agent that detects and kills Bacillus anthracis. Nature, 418: 884-889.
Spencer, R. C. & Lightfoot, N. F. (2001). Preparedness and response to bioterrorism. J Infect, 43: 104-10.
Strong, L., Yarmush, D. M. & Yarmush, M. L. (1994). Antibody-targeted photolysis. Photophysical, biochemical, and pharmacokinetic properties of antibacterial conjugates. [Review]. Ann N Y Acad Sci, 745: 297-320.
Trauner, K. B. & Hasan, T. (1996). Photodynamic treatment of rheumatoid and inflammatory arthritis. Photochem Photobiol, 64: 740-50.
Tutrone, W. D., Scheinfeld, N. S. & Weinberg, J. M. (2002). Cutaneous anthrax: a concise review. Cutis, 69: 27-33.
Valduga, G., Breda, B., Giacometti, G. M., Jori, G. & Reddi, E. (1999). Photosensitization of wild and mutant strains of Escherichia coli by meso-tetra(N-methyl-4-pyridyl)porphine. Biochem Biophys Res Commun, 256: 84-8.
Von Tappeiner, H. & Jodlbauer, A. (1904). Ueber wirkung der photodynamischen (fluorescierenden) Stoffe auf Protozoan und enzyme. Disch. Arch. Klin. Med., 80: 422-487.
Whitney, E. S., Beatty, M. E., Taylor, T. H., Jr, Weyant, R., Sobel, J., Arduino, M. J. & Ashford, D. A. (2003). Inactivation of Bacillus anthracis Spores. Emerg. Infect. Dis., 9: 623-627.
Wiener, S. L. (1996). Strategies for the prevention of a successful biological warfare aerosol attack. Mil Med, 161: 251-6.
Wilson, M. (1993). Photolysis of oral bacteria and its potential use in the treatment of caries and periodontal disease. J. Appl. Bacteriol., 75: 299-306.
Wilson, M. & Pratten, J. (1995). Lethal photosensitisation of Staphylococcus aureus in vitro: effect of growth phase, serum, and pre-irradiation time. Lasers Surg Med, 16: 272-6.
Wilson, M. & Yianni, C. (1995). Killing of methicillin-resistant Staphylococcus aureus by low-power laser light. J Med Microbiol, 42: 62-6.
Yudkin, M. (1993). Spore formation in Bacillus subtilis. Sci Prog, 77: 113-30.
Ziebell, R., Imray, F. P. & Macphee, D. G. (1977). DNA degradation in wild-type and repair-deficient strains of salmonella typhimurium exposed to ultraviolet light of photodynamic treatment. J Gen Microbiol, 101: 143-9.

1. A method of inactivating bacterial spores comprising contacting the bacterial spores with a photosensitizer and irradiating the bacterial spores such that a phototoxic species is produced that inactivates the bacterial spores.

2. The method of claim 1 wherein the bacterial spores are produced by bacteria of the genus Bacillus.

3. The method of claim 2 wherein the bacterial spores are produced by bacteria of the species selected from the group consisting of Bacillus anthracis, Bacillus cereus, Bacillus thuringiensis, Bacillus subtilis and Bacillus atrophaeus.

4. The method of claim 1 wherein the bacterial spores are produced by bacteria of the genera selected from the group consisting of Clostridium, Methylosinus, Azotobacter, Bdellovibrio, Myxococcus, Cyanobacteria, Thermoactinomyces, Myxococcus, Desulfotomaculum, Marinococcus, Sporosarcina, Sporolactobacillus and Oscillospira.

5. The method of claim 1, wherein the bacterial spores to be inactivated are located in or on a living animal.

6. The method of claim 5 wherein the bacterial spores to be inactivated are located on the skin or mucous membranes of the living animal, or within wounds, cuts or abrasions in the skin or mucous membranes of the living animal.

7. The method of claim 5 or 6 wherein the living animal is a human.

8. The method of claim 1, wherein the bacterial spores to be inactivated are located in or on an inanimate object or substance.

9. The method of claim 8, wherein the inanimate object or substance comprises a surface, a fluid or a gas.

10. The method of claim 1, wherein the photosensitizer is selected from the group consisting of phenothiazinium dyes, phenodiazinium dyes, phenooxazinium dyes, and mixtures thereof.

11. The method of claim 1, wherein the photosensitizer is selected from the group consisting of phenothiazinium, phenodiazinium, phenoselenazinium and mixtures thereof.

12. The method of claim 1, wherein the photosensitizer is selected from the group consisting of toluidine blue derivatives, toluidine blue O, methylene blue, new methylene blue N, new methylene blue BB, new methylene blue FR, 1,9-dimethylmethylene blue chloride, methylene blue derivatives, methylene green, methylene violet Bernthsen, methylene violet 3RAX, Nile blue, Nile blue derivatives, malachite green, Azure blue A, Azure blue B, Azure blue C, safranine 0, neutral red, 5-ethylamino-9-diethylaminobenzo[a]phenothiazinium chloride, 5-ethylamino-9-diethylaminobenzo[a]phenoselenazinium chloride, thiopyronine, thionine, and mixtures thereof.

13. The method of claims 10, wherein the bacterial spores are contacted with a composition comprising the photosensitizer.

14. The method of claims 13, wherein the composition further comprises a member selected from the group consisting of a pharmaceutically acceptable carrier, an excipient, an antibiotic, a sporicidal agent, a disinfectant, and a detergent.

15. The method of claim 1, wherein the method further comprises contacting the bacterial spores with an antibiotic, a sporicidal agent, a disinfectant, or a detergent.

16. The method of claim 15, wherein the bacterial spores are contacted with the photosensitizer and the antibiotic, sporicidal agent, disinfectant, or detergent at the same time.

17. The method of claim 15, wherein the bacterial spores are contacted with the photosensitizer before they are contacted by the antibiotic, sporicidal agent, disinfectant, or detergent.

18. The method of claim 15, wherein the bacterial spores are contacted with the photosensitizer after they are contacted by the antibiotic, sporicidal agent, disinfectant, or detergent.

19. The method of claim 13, wherein the photosensitizer composition comprises a liquid, cream, or lotion.

20. The method of claim 13, wherein the photosensitizer composition comprises a liquid spray.

21. The method of claim 13 wherein the photosensitizer composition comprises an aerosol spray.

22. The method of claim 1, wherein the irradiation is provided by a light source that emits light at wavelength in the range of about 450 to about 750 nm

23. The method of claim 1, wherein the irradiation is provided by a light source that emits light at fluence in the range of about 10 to about 1000 J/cm²

24. The method of claim 1, wherein the irradiation is provided by a light source that emits light at wavelength in the range of about 450 to about 750 nm and a fluence in the range of about 10 to about 1000 J/cm².

25. The method of claim 1, wherein the irradiation is provided by a lamp, a laser or a fiber optic device.

26. A method of treating a subject contaminated with bacterial spores, said method comprising the steps of administering a photosensitizer to the subject, irradiating the subject such that a phototoxic species is produced that inactivates the bacterial spores, thereby treating the subject.

27. The method of claim 26, wherein the bacterial spores are produced by bacteria of the genus Bacillus.

28. The method of claim 25, wherein the bacterial spores are produced by bacteria of the species selected from the group consisting of Bacillus anthracis, Bacillus cereus, Bacillus thuringiensis, Bacillus subtilis and Bacillus atrophaeus.

29. The method of claim 26, wherein the bacterial spores are produced by bacteria of the genera selected from the group consisting of Clostridium, Methylosinus, Azotobacter, Bdellovibrio, Myxococcus, Cyanobacteria, Thermoactinomyces, Myxococcus, Desulfotomaculum, Marinococcus, Sporosarcina, Sporolactobacillus and Oscillospira.

30. The method of claim 26, wherein the bacterial spores to be inactivated are located in or on the subject.

31. The method of claim 28, wherein the bacterial spores to be inactivated are located on the skin or mucous membranes of the subject, or within wounds, cuts or abrasions in the skin or mucous membranes of the subject.

32. The method of claim 26, wherein the subject is a human.

33. The method of claim 26, wherein the photosensitizer is selected from the group consisting of phenothiazinium dyes, phenodiazinium dyes, phenooxazinium dyes and mixtures thereof.

34. The method of claim 26, wherein the photosensitizer is selected from the group consisting of phenothiazinium, phenodiazinium, phenoselenazinium and mixtures thereof.

35. The method of claim 26, wherein the photosensitizer is selected from the group consisting of, toluidine blue derivatives, toluidine blue O, methylene blue, new methylene blue N, new methylene blue BB, new methylene blue FR, 1,9-dimethylmethylene blue chloride, methylene blue derivatives, methylene green, methylene violet Bemthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, thionine and mixtures thereof.

36. The method of claims 33, wherein a composition comprising the photosensitizer is administered to the subject.

37. The method of claim 36, wherein the composition further comprises a member selected from the group consisting of a pharmaceutically acceptable carrier, an excipient, an antibiotic, a sporicidal agent, a disinfectant, and a detergent.

38. The method of claim 26, wherein the method further comprises administering an antibiotic or a sporicidal agent.

39. The method of claim 35, wherein the antibiotic or sporicidal agent is administered at the same time as the photosensitizer.

40. The method of claim 35, wherein the antibiotic or sporicidal agent is administered before the photosensitizer.

41. The method of claim 35, wherein the antibiotic or sporicidal agent is administered after the photosensitizer composition.

42. The method of claim 36 wherein the photosensitizer composition comprises a liquid, cream, or lotion.

43. The method of claim 36, wherein the photosensitizer composition comprises a liquid spray.

44. The method of claim 36, wherein the photosensitizer composition comprises an aerosol spray.

45. The method of claim 26, wherein the irradiation is provided by a light source that emits light at wavelength in the range of about 450 to about 750 mm.

46. The method of claim 26, wherein the irradiation is provided by a light source that emits light at fluence in the range of about 10 to about 1000 J/cm².

47. The method of claim 26, wherein the irradiation is provided by a light source that emits light at wavelength in the range of about 450 to about 750 nm and a fluence in the range of about 10 to about 1000 J/cm².

48. The method of claim 26, wherein the irradiation is provided by a lamp, a laser or a fiber optic device.

49. The method of claims 1, further comprising obtaining the photosensitizer.

50. The method of claims 1, further comprising synthesizing the photosensitizer.

51. The method of claims 13, further comprising obtaining the composition.

52. The method of claims 13, further comprising synthesizing the composition.

53. The method of claim 26, wherein the step of administering comprises topical application of the photosensitizer.

54. The method of claim 26, wherein the step of administering comprises inhalation of the photosensitizer.

55. The method of claim 26, wherein the step of administering comprises ingestion of the photosensitizer.

56. The method of claim 26, wherein the step of administering comprises injection of the photosensitizer.

57. The method of claim 26, wherein the step of administering comprises implantation of the photosensitizer.

58. A kit for inactivating bacterial spores comprising a photosensitizer and directions for use.

59. The kit of claim 58, further comprising means for irradiating the bacterial spores.

60. The kit of claim 58, wherein the photosensitizer as a phtosensitizer composition.

61. A kit for treating a subject contaminated with bacterial spores comprising a photosensitizer and instructions for use.

62. The kit of claim 61, further comprising means for irradiating the subject.

63. The kit of claim 61, wherein the photosensitizer is present in a composition comprising a therapeutically effective amount of the photosensitizer.
